(19) United States
(12) Patent Application Publication
(10) Pub. No.: US 2026/0308116 A1
(43) Pub. Date: Oct. 8, 2026

(54) TERTIARY AMINE COMPOUNDS CONTAINING AN ALKYNE OR AN AZIDE MOIETY AND THEIR USE

(71) Applicants: INSTITUT NATIONAL DE LA SANTÉ ET DE LA RECHERCHE MÉDICALE, PARIS (FR); UNIVERSITE PARIS CITE, PARIS (FR); ASSISTANCE PUBLIQUE HOPITAUX DE PARIS, PARIS (FR); INSTITUT JEAN PAOLI & IRENE CALMETTES, MARSEILLE (FR); UNIVERSITÉ D'AIX-MARSEILLE, MARSEILLE (FR); CENTRE NATIONAL DE LA RECHERCHE SCIENTIFIQUE, PARIS (FR)

(72) Inventors: Christophe LACHAUD, MARSEILLE CEDEX 09 (FR); Jean SOULIER, PARIS (FR); Sébastien COMBES, MARSEILLE CEDEX 5 (FR); Sara BERRADA, QUEBEC (CA); Norbert VEY, MARSEILLE (FR)

(21) Appl. No.: 19/481,680

(22) PCT Filed: May 3, 2024
(86) PCT No.: PCT/EP2024/062330

§ 371 Date: Nov. 5, 2025

(30) Foreign Application Priority Data
May 5, 2023 (EP) 23315169.5

(51) Int. Cl. C07C 237/20; C07C 247/04; G01N 33/50; G01N 33/58

(52) U.S. Cl. CPC C07C 237/20; C07C 247/04; G01N 33/502; G01N 33/582

(57) ABSTRACT

Tertiary amine compounds that have an alkyne group or an azide group, in particular an alkyne group, and/or their pharmaceutically acceptable salts and solvates that are useful to monitor DNA lesions and to reveal interstrand crosslinks repair defects are disclosed herein. Methods of monitoring DNA lesions in patients selected from Fanconi Anemia (FA) patients, xeroderma pigmentosum (XP) patients, acute myeloid leukemia (AML) patients, head-and-neck cancer patients, ovarian cancer patients, and breast cancer patients using the tertiary amine compounds and/or their pharmaceutically acceptable salts and solvates are also disclosed.

The present invention relates to tertiary amine compounds, containing an alkyne group or an azide group, in particular an alkyne group, including their pharmaceutically acceptable salts and solvates, which are useful to monitor DNA lesions and to reveal interstrand crosslinks repair defects, in particular in patients selected from Fanconi Anemia (FA) patients, xeroderma pigmentosum (XP) patients, acute myeloid leukemia (AML) patients, head-and-neck cancer patients, ovarian cancer patients and breast cancer patients.

### BACKGROUND OF THE INVENTION

Fanconi anemia (FA) is an autosomal recessive disease characterized by genomic instability (Shimamura et al., Blood Rev., 2010, 24, 101-122). FA may be suspected at birth or in young children by the presence of one or more of physical traits (including short stature, hand, arm and other skeletal anomalies, kidney defects, small head or eyes). However, since some patients show no obvious physical traits, FA is often diagnosed only when a cytopenia is detected. The usual period of development of MDS/AML is the late teenage years or young adulthood. FA is associated with an increased susceptibility to develop myelodysplastic syndromes (MDS) and leukemias, especially acute myeloid leukemias (AML). AML in the setting of FA is uneasy to treat and is associated with a bad prognosis (Peffault de Latour et al., Blood, 2016, 127, 2971-2979). Patients with FA also have a high susceptibility to develop specific solid malignancies, especially head and neck cancers (Rosenberg et al., Blood, 2005, 105, 67-73). Thus, FA patients require increased surveillance for hematologic and non-hematologic malignancies and other organs dysfunction.

Determining whether FA is the cause of bone marrow failure or AML when they are detected in an individual has important implications for the patient management. Indeed, FA patients should receive reduced doses of chemotherapy for treating malignancies and in the preparative regimen for hematopoietic cell transplantation (Peffault de Latour et al., Blood, 2013, 122, 4279-4286; Peffault de Latour et al., Blood, 2016, 127, 2971-2979). FA is due to loss of function mutations in genes encoding proteins of the FA/BRCA pathway, that are involved in DNA repair (Ceccaldi et al., Nat. Rev. Mol. Cell Biol., 2016, 17, 337-349). The major described role of the FA/BRCA pathway is the processing of interstrand crosslinks (ICLs) at stalled replication forks. An ICL is a covalent link established between the two strands of the DNA, which blocks replication. ICLs can be induced by endogenous molecules such as aldehydes or by exogenous genotoxins such as alkylating agents.

Due to a high number of FANC genes and many private mutations difficult to distinguish from variant of unknow significance (VUS), and given the possible reversion of phenotype in hematopoietic cells, it is not feasible to diagnose FA upfront using direct DNA sequencing test, even with the described gene panel or whole genome approaches. The usual method to diagnose FA remains the chromosome breakage test upon ICLs exposure. While efficient, this is a labour-intensive and costly method. In addition, the chromosomal breakage test is often non conclusive in two situations: 1/At MDS or AML diagnosis when an underlying FA is suspected due to physical or biological signs or an unusual young age (in MDS patients); 2/After a first chemotherapy when FA is suspected due to an unexpectedly high toxicity. In these two situations, the classical breakage tests are often inconclusive because blast cells behave differently than lymphocytes and/or due to confounding breaks induced by prior chemotherapy, respectively. The direct functional analysis of the FA/BRCA pathway is faster than the chromosome break test but suffers the same limitation to diagnose patients at the MDS/AML stage (Soulier et al., Blood, 2005, 105, 1329-1336). Alternative methods have thus been developed to enable the analysis of ICLs hypersensitivity in primary fibroblast cells, such as dedicated flow cytometry, but these are still labour-intensive and costly (Pinto et al., Haematologica, 2009, 94, 487-495).

All the current methods for the diagnosis of FA are based on the detection of unrepaired ICL lesions consequences. One solution to overcome all the existing limitations would be to directly quantify the accumulation of ICL-inducing agents on the DNA. Psoralen is a DNA intercalating agent that can be photoactivated by UVA to generate almost exclusively ICLs (Dunnick et al., Toxicol. Appl. Pharmacol., 1987, 89, 73-80). At least two detectable psoralen have been generated (Evison et al., Bioorg. Med. Chem., 2016, 24, 1071-1078; Muniandy et al., J. Biol. Chem., 2009, 284, 27908-27917) allowing the quantification of psoralen accumulation. However, since psoralen-induced ICLs are not preferentially repaired by FA (Semlow et al., Cell, 2016, 167, 498-511.e14; Li et al., Nucleic Acids Res., 2020, 11, 467) the use of psoralen to diagnose FA is not appropriate. Other detectable ICL inducing agent derivate of platinum compound exist (White et al., J. Am. Chem. Soc., 2013, 135, 11680-11683; Osborn et al., ACS Chem. Biol., 2014, 9, 2404-2411; Ding et al., Angew. Chem. Int. Ed. Engl., 2013, 52, 3350-3354; Qiao et al., J. Biol. Inorg. Chem., 2014, 19, 415-426; Zacharioudakis et al., Angew. Chem. Int. Ed. Engl., 2017, 56, 6483-6487). However, these agents mainly generate intrastrand crosslinks and ICLs only represent a small fraction of all lesions (Eastman, Biochemistry, 1986, 25, 3912-3915). Therefore, the detection of the accumulation of these ICL-inducing agents will not automatically represent ICLs accumulation.

There is thus a need for a method for differentially monitoring ICLs from lesions affecting only one DNA strand for crosslinking agents that do not produce a majority of ICLs.

### SUMMARY OF THE INVENTION

The inventors have now succeeded in developing tertiary amine compounds, containing an alkyne group or an azide group, in particular an alkyne group. These compounds have the advantage of enabling detection of DNA crosslinks lesions induced by alkylating agents in cells. In particular, the compounds of the invention have similar activities compared to unmodified melphalan and can be used to differentially monitor lesion accumulation and repair in cells during the different cell cycle phases. The compounds of the invention may thus be used for the detection of ICL repair defects in patients selected from Fanconi Anemia (FA) patients, xeroderma pigmentosum (XP) patients, acute myeloid leukemia (AML) patients, head-and-neck cancer patients, ovarian cancer patients and breast cancer patients, in particular Fanconi Anemia (FA) patients and xeroderma pigmentosum (XP) patients. In addition, the compounds of the present invention are less toxic than compounds for visualizing DNA crosslinks of the prior art, in particular pyrrolo[2,1-c][1,4]benzodiazepines (PBDs) of WO 2021/074392.

The invention therefore relates to compounds of general Formula I, their pharmaceutically acceptable salts and solvates as well as methods of use of such compounds.

In a general aspect, the invention provides compounds of general Formula I:

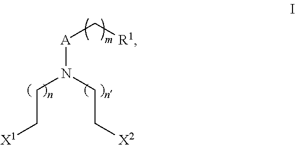

a pharmaceutically acceptable salt or a solvate thereof,
wherein
R¹ is C≡CH or N₃;
m is an integer ranging from 1 to 6,
A is selected from

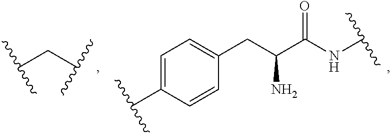

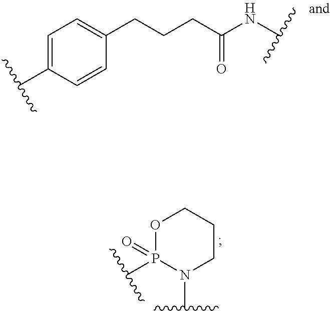 [SMILES: *NC(=O)CCCc1ccc(*)cc1 ; *N1CCCOP1(*)=O]

n and n′ are integers independently ranging from 1 to 6;
X¹ is halogen; and
X² is halogen or OH.

In another aspect, the present invention provides a kit comprising at least one compound according to the invention, and at least one label bearing a group complementary to the R¹ group of said at least one compound for a “click chemistry” reaction, the label being in particular a fluorescent label or a biotinylated label.

The invention also relates to an in-vitro or ex vivo method for detecting DNA crosslinks lesions induced by alkylating agents in cells, the method comprising:
(a) having at least one cell,
(b) contacting said cell with at least one compound of formula (I) under conditions allowing said compound to induce DNA crosslinks in the cell,
(c) contacting the cell obtained in step (b) with at least one label bearing a group which is complementary for a click-chemistry reaction to the R¹ group of the said compound of formula (I), under conditions allowing the reaction of click-chemistry between the said R¹ group and the complementary group,
(d) detecting the label in the cell obtained at step (c).

The invention also relates to an in vitro or ex vivo method for detecting ICL repair defects in a patient cells, comprising at least the steps of:
(a) having at least one cell,
(b) contacting said cell with at least one said compound of formula (I) under conditions allowing said compound to induce DNA crosslinks in the cell,
(c) contacting the cell obtained in step (b) with at least one label bearing a group which is complementary for a click-chemistry reaction to the R¹ group of the compound of formula (I) under conditions allowing the reaction of click-chemistry between the said R¹ group and the complementary group,
(d) measuring the labeling in the cell obtained at step (c), and
(e) optionally comparing the labeling measured at step (d) to a reference level.

### DETAILED DESCRIPTION OF THE INVENTION

### Compounds of the Invention

As detailed above, the invention relates to compounds of Formula I, as well as their pharmaceutically acceptable salts or solvates.

Preferred compounds of Formula I or pharmaceutically acceptable salts or solvates thereof are those wherein one or more of R¹, m, A, n, n′, X¹ and X² are defined as follows:
R¹ is C≡CH or N₃; in particular R¹ is C≡CH;
m is an integer ranging from 1 to 6; in particular m is an integer ranging from 1 to 4; more particularly m is an integer ranging from 1 to 3, still more particularly m is 1 or 2; even more particularly m is 1; for example m is 1 or 3;
A is selected from

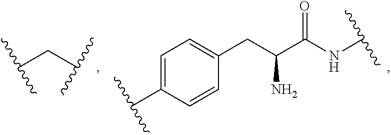

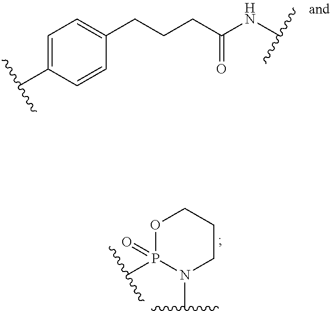 [SMILES: *NC(=O)CCCc1ccc(*)cc1 ; *N1CCCOP1(*)=O]

in particular A is selected from

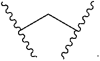 [SMILES: *C*]

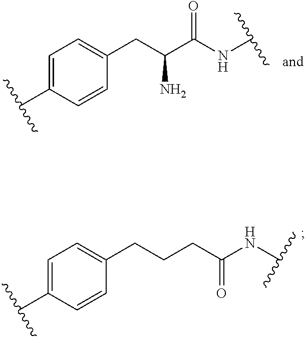 [SMILES: *NC(=O)[C@@H](N)Cc1ccc(*)cc1 ; *NC(=O)CCCc1ccc(*)cc1]

more particularly A is

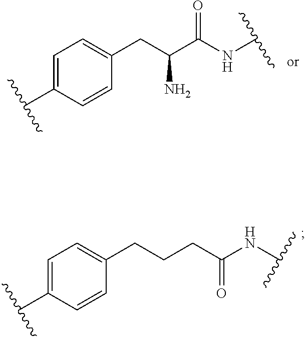 [SMILES: *NC(=O)[C@@H](N)Cc1ccc(*)cc1 ; *NC(=O)CCCc1ccc(*)cc1]

still more particularly A is

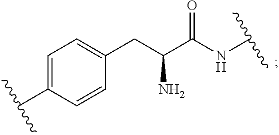 [SMILES: *NC(=O)[C@@H](N)Cc1ccc(*)cc1]

for example A is selected from

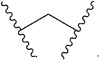 [SMILES: *C*]

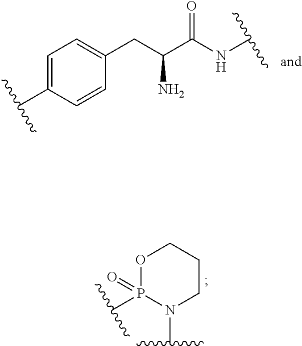 [SMILES: *NC(=O)[C@@H](N)Cc1ccc(*)cc1 ; *N1CCCOP1(*)=O]

in a particular example A is

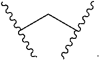 [SMILES: *C*]

or

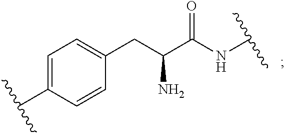 [SMILES: *NC(=O)[C@@H](N)Cc1ccc(*)cc1]

in a more particular example A is

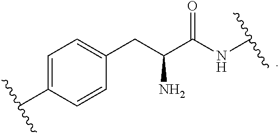 [SMILES: *NC(=O)[C@@H](N)Cc1ccc(*)cc1]

n and n′ are integers independently ranging from 1 to 6; in particular n and n′ are integers independently ranging from 1 to 4; more particularly n and n′ are integers independently ranging from 1 to 3; still more particularly n and n′ are independently 1 or 2; even more particularly n and n′ are 1;
X¹ is halogen; in particular X¹ is selected from F, Cl, Br and I; more particularly X¹ is selected from F, Cl and Br; still more particularly X¹ is F or Cl; even more particularly X¹ is Cl;
X² is halogen or OH; in particular X² is selected from F, Cl, Br, I and OH; more particularly X² is selected from F, Cl, Br and OH; still more particularly X² is selected from F, Cl and OH; even more particularly X² is Cl or OH.

Halogens include a fluorine atom (F), an iodine atom (I), a chlorine atom (Cl) and a bromine atom (Br).

In one embodiment, the compounds of Formula I are those wherein R¹ is C≡CH.

In one embodiment, the compounds of Formula I are those wherein R¹ is N₃.

In one embodiment, the compounds of Formula I are those wherein m is an integer ranging from 1 to 3.

In one embodiment, the compounds of Formula I are those wherein m is 1.

In one embodiment, the compounds of Formula I are those wherein m is 3.

In one embodiment, the compounds of Formula I are those wherein A is

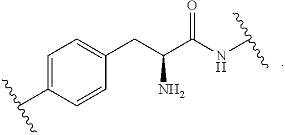 [SMILES: *NC(=O)[C@@H](N)Cc1ccc(*)cc1]

In one embodiment, the compounds of Formula I are those wherein A is

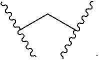 [SMILES: *C*]

In one embodiment, the compounds of Formula I are those wherein A is

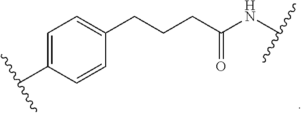 [SMILES: *NC(=O)CCCc1ccc(*)cc1]

In one embodiment, the compounds of Formula I are those wherein A is

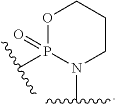 [SMILES: *N1CCCOP1(*)=O]

In one embodiment, the compounds of Formula I are those wherein n and n′ are 1.

In one embodiment, the compounds of Formula I are those wherein X¹ is Cl.

In one embodiment, the compounds of Formula I are those wherein X² is Cl or OH.

In one embodiment, the compounds of Formula I are those wherein A is

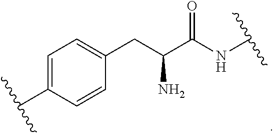 [SMILES: *NC(=O)[C@@H](N)Cc1ccc(*)cc1]

The compounds according to this embodiment are thus those of Formula II:

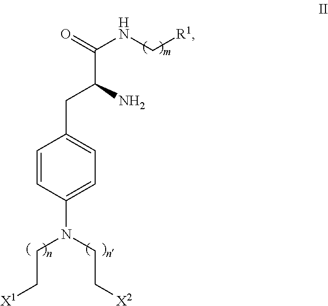

or pharmaceutically acceptable salts or solvates thereof,
wherein
R¹, m, n, n′, X¹ and X² are as defined above with respect to Formula I and any of its embodiments.

In one embodiment, the compounds of Formula II are those wherein R¹ is C≡CH.

In one embodiment, the compounds of Formula II are those wherein R¹ is N₃.

In one embodiment, the compounds of Formula II are those wherein m is an integer ranging from 1 to 3.

In one embodiment, the compounds of Formula II are those wherein m is 1.

In one embodiment, the compounds of Formula II are those wherein m is 3.

In one embodiment, the compounds of Formula II are those wherein n and n′ are 1.

In one embodiment, the compounds of Formula II are those wherein X¹ is Cl.

In one embodiment, the compounds of Formula II are those wherein X² is Cl or OH.

In one embodiment, the compounds of Formula I are those wherein R¹ is C≡CH. The compounds according to this embodiment are thus those of Formula III:

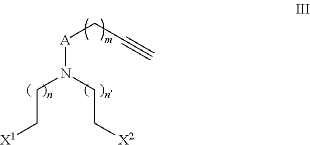

or pharmaceutically acceptable salts or solvates thereof,
wherein
m, A, n, n′, X¹ and X² are as defined above with respect to Formula I and any of its embodiments.

In one embodiment, the compounds of Formula III are those wherein m is an integer ranging from 1 to 3.

In one embodiment, the compounds of Formula III are those wherein m is 1.

In one embodiment, the compounds of Formula III are those wherein m is 3.

In one embodiment, the compounds of Formula III are those wherein A is

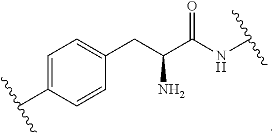 [SMILES: *NC(=O)[C@@H](N)Cc1ccc(*)cc1]

In one embodiment, the compounds of Formula III are those wherein n and n′ are 1.

In one embodiment, the compounds of Formula III are those wherein X¹ is Cl.

In one embodiment, the compounds of Formula III are those wherein X² is Cl or OH.

In one embodiment, the compounds of Formula I are those wherein R¹ is N₃. According to this embodiment, the compounds of Formula I are those of Formula IV:

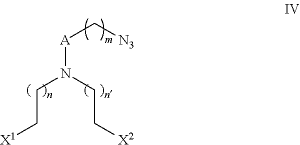

or pharmaceutically acceptable salts or solvates thereof,
wherein
m, A, n, n′, X¹ and X² are as defined above with respect to Formula I and any of its embodiments.

In one embodiment, the compounds of Formula IV are those wherein m is an integer ranging from 1 to 3.

In one embodiment, the compounds of Formula IV are those wherein m is 1.

In one embodiment, the compounds of Formula IV are those wherein m is 3.

In one embodiment, the compounds of Formula IV are those wherein A is

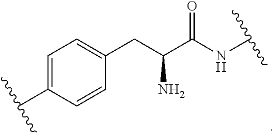 [SMILES: *NC(=O)[C@@H](N)Cc1ccc(*)cc1]

In one embodiment, the compounds of Formula IV are those wherein n and n′ are 1.

In one embodiment, the compounds of Formula IV are those wherein X¹ is Cl.

In one embodiment, the compounds of Formula IV are those wherein X² is Cl or OH.

In one embodiment, the compounds of Formula II are those wherein R¹ is C≡CH. The compounds according to this embodiment are thus those of Formula V:

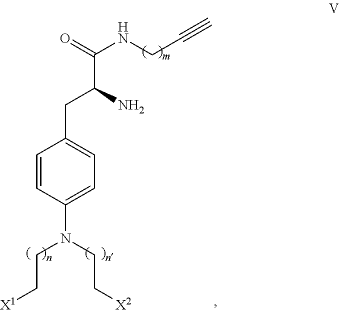

or pharmaceutically acceptable salts or solvates thereof,
wherein
m, n, n′, X¹ and X² are as defined above with respect to Formula I and any of its embodiments.

In one embodiment, the compounds of Formula V are those wherein m is an integer ranging from 1 to 3.

In one embodiment, the compounds of Formula V are those wherein m is 1.

In one embodiment, the compounds of Formula V are those wherein m is 3.

In one embodiment, the compounds of Formula V are those wherein n and n′ are 1.

In one embodiment, the compounds of Formula V are those wherein X¹ is Cl.

In one embodiment, the compounds of Formula V are those wherein X² is Cl or OH.

In one embodiment, the compounds of Formula II are those wherein R¹ is N₃. The compounds according to this embodiment are thus those of Formula VI:

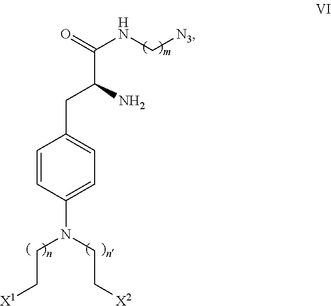

or pharmaceutically acceptable salts or solvates thereof,
wherein
m, n, n′, X¹ and X² are as defined above with respect to Formula I and any of its embodiments.

In one embodiment, the compounds of Formula VI are those wherein m is an integer ranging from 1 to 3.

In one embodiment, the compounds of Formula VI are those wherein m is 1.

In one embodiment, the compounds of Formula VI are those wherein m is 3.

In one embodiment, the compounds of Formula VI are those wherein n and n′ are 1.

In one embodiment, the compounds of Formula VI are those wherein X¹ is Cl.

In one embodiment, the compounds of Formula VI are those wherein X² is Cl or OH.

Particularly preferred compounds of the invention are those listed in Table 1 hereafter:

TABLE 1

| Compound | Structure | Name |
|---|---|---|
| 1 | 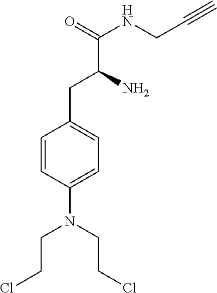 [SMILES: C#CCNC(=O)[C@@H](N)Cc1ccc(N(CCCl)CCCl)cc1] | (S)-2-amino-3-(4-(bis(2- chloroethyl)amino)phenyl)-N-(prop-2-yn-1- yl)propanamide |
| 2 | 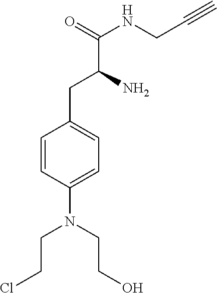 [SMILES: C#CCNC(=O)[C@@H](N)Cc1ccc(N(CCO)CCCl)cc1] | (S)-2-amino-3-(4-((2-chloroethyl)(2- hydroxyethyl)amino)phenyl)-N-(prop-2-yn-1- yl)propanamide |
| 3 | 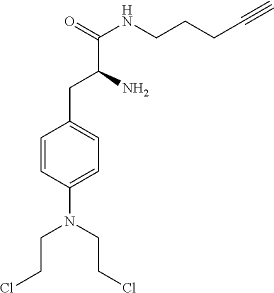 [SMILES: C#CCCCNC(=O)[C@@H](N)Cc1ccc(N(CCCl)CCCl)cc1] | (S)-2-amino-3-(4-(bis(2- chloroethyl)amino)phenyl)-N-(pent-4-yn-1- yl)propanamide |
| 4 | 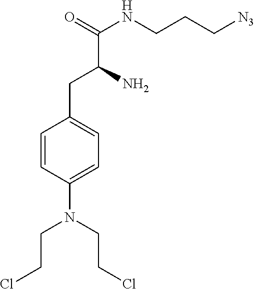 [SMILES: [N-]=[N+]=NCCCNC(=O)[C@@H](N)Cc1ccc(N(CCCl)CCCl)cc1] | (S)-2-amino-N-(3-azidopropyl)-3-(4-(bis(2- chloroethyl)amino)phenyl)propanamide |

The compounds of the invention can be prepared by different ways with reactions known by the person skilled in the art, in particular as described below in the “Examples” section.

In particular, compound 1 of the Table 1 above (compound 4 of Example 1) may be prepared in three steps from commercially available melphalan ((S)-2-amino-3-(4-(bis(2-chloroethyl)amino)phenyl)-N-(prop-2-yn-1-yl) propenamide):
(i) protection of the primary amine, in particular with di-tert-butyl dicarbonate;
(ii) reaction of the product obtained in step (i) with propargyl amine, in particular in the presence of a peptidic coupling agent, more particularly in the presence of benzotriazol-1-yloxytris(dimethylamino)phosphonium hexafluorophosphate (BOP);
(iii) removal of the N-protecting group.

In particular, compound 2 of the Table 1 above (compound 6 of Example 2) may be prepared in one step from compound 1 of the Table 1 above (compound 4 of Example 1) by hydrolysis with chlorhydric acid.

The present invention also relates to a composition comprising a compound of formula (I) as provided herein above.

### Kit According to the Invention

The compounds of formula (I) according to the present invention are suitable for forming detectable DNA crosslinks.

Thus, the present invention provides a kit useful for performing labelled DNA crosslinks using compounds of formula (I) according to the invention.

In particular, the present invention provides a kit comprising:
at least a compound of formula (I) as previously defined; and
at least a label bearing a group complementary to the R¹ radical of the compound of formula (I) for a “click-chemistry” reaction, i.e. when the R¹ radical of the compound of formula (I) is an alkyne group, then the complementary group borne by the label is an azide group.

Alternatively, when the R¹ radical of the compound of formula (I) is an azide group, then the complementary group borne by the label is an alkyne group.

A label comprised in a kit according to the invention can be a moiety that is directly or indirectly detectable. In particular, a label can be selected from dyes, radiolabels and affinity tags.

More particularly, dyes can be selected from the group consisting of fluorescent, luminescent or phosphorescent dyes, preferably dansyl, fluorescein, acridine, rhodamine, coumarine, BODIPY and cyanine dyes.

In a particular embodiment, a kit as provided herein comprises a label that is a fluorescent dye.

A fluorescent dye comprised in a kit according to the invention can be selected among the dyes marketed by Molecular Probes™ such as the Alexa Fluor dyes, Pacific dyes or Texas Red or by other providers for cyanines 3, 5 and 7. In particular, dyes bearing an alkyne or an azide group are commercially available. For example, the fluorescent dye comprised in a kit according to the invention can be selected among Alexa Fluor™ 488, 555, 594 and 647 and for TAMRA (tetramethylrhodamine). In a particular example, the fluorescent dye comprised in a kit according to the invention may be Alexa Fluor™ 647.

Alternatively, a label comprised in a kit as provided herein can be an affinity tag.

Such label can be, for instance, selected from the group consisting of biotin, His-tag, Flag-tag, strep-tag, sugars, lipids, sterols, PEG-linkers and co-factors. More particularly, said label is a biotinylated label, especially a biotinylated polyethylene glycol label such as Biotin-PEG4 alkyne or other biotins linked to an alkyne or an azide that are commercially available such as Biotin DIBO Alkyne sold by Molecular Probes™.

Finally, a label comprised in a kit as provided herein can be a radiolabel.

Such label can be, for instance, selected from the group consisting of radioactive forms of hydrogen, carbon, phosphorus, Sulphur and iodine. More particularly, said label can be selected from the group consisting of tritium, carbon-11, carbon-14, phosphorus-32, phosphorus-33, Sulphur-33, iodine-123 and iodine-125.

In a particular embodiment, the label is a fluorescent label or a biotin. In a more particular embodiment, the label is a fluorescent label.

A kit as provided herein may further comprise at least one agent selected from the group consisting of copper, more particularly copper (II) precursor with a reducing agent, copper (I) salts or pre-formed copper (I) complexes; a permeabilizing agent; a fixation solution; a washing buffer; and a leaflet comprising explanation for the use of the kit.

In a preferred embodiment, the copper reagent is preferably copper (II) with sodium ascorbate. The permeabilizing agent is a cytoskeleton (CSK) buffer comprising Triton X-100 or any equivalent buffer comprising a detergent suitable for permeabilizing eukaryotic cell membrane. The fixation solution contains paraformaldehyde (PFA) or any equivalent known by the person skilled in the art. The washing buffer is phosphate-buffered saline (PBS) or any equivalent known by the person skilled in the art.

The present invention also relates to several methods implementing a kit or a compound of the invention.

### Uses and Methods According to the Present Invention

As previously mentioned, a compound of formula (I) of the invention, a composition comprising said compound, or a kit of the invention as defined above, is particularly useful as a research tool.

According to a first aspect, a compound of formula (I) according to the invention enables in vitro or ex vivo detection and visualization of DNA crosslinks in a cell. This labeling allows the localization, quantification or isolation of DNA crosslinks.

Accordingly, the present invention relates to an in vitro or ex vivo method for detecting DNA crosslinks lesions induced by alkylating agents in cells, the method comprising:
(a) having at least one cell;
(b) contacting said cell with at least one compound of formula (I) according to the invention under physiological conditions allowing said compound to induce DNA crosslinks in the cell;
(c) contacting the cell obtained in step (b) with at least one label bearing a group which is complementary for a click-chemistry reaction to the R¹ group of the compound of formula (I) under conditions allowing the reaction of click-chemistry between said R¹ group and the complementary group;
(d) detecting the label in the cell obtained at step (c).

Said label can in particular be a fluorescent label.

In particular the alkylating agent may be selected from melphalan, chlorambucil, chlormethine and cyclophosphamide, more particularly the alkylating agent may be selected from melphalan, chlorambucil and chlormethine, still more particularly the alkylating agent may be selected from melphalan and chlorambucil, even more particularly the alkylating agent may be melphalan.

Physiological conditions mean that the considered steps are not carried out in extreme conditions (pH, temperature, pression . . . ) that would lead to a degradation of the cells and/or compounds involved in a method according to the invention.

Step (c) of this method can be performed after or simultaneously to step (b).

In an embodiment where step (b) is performed before step (c), the at least one cell can be incubated with the candidate molecule and/or exposed to the candidate treatment during a period ranging from 1 hour to 5 days, preferably from 1 hour to 4 days, for example from 1 hour to 3 days, from 1 hour to 2 days, from 1 hour to 24 hours, from 1 hour to 12 hours.

In particular, the method can comprise a step of cell membrane permeabilization, in particular a pre-extraction treatment with CSK buffer or PBS triton 0.5%, optionally followed by a step of fixation, between steps (b) and (c). The method can alternatively comprise a step of cell membrane permeabilization, optionally followed by a step of fixation, between steps (c) and (d). According to a particular embodiment, the method comprises a step of cell membrane permeabilization, optionally followed by a step of fixation, between steps (b) and (c) and a step of cell membrane permeabilization, optionally followed by a step of fixation, between steps (c) and (d).

Performing a permeabilization step before the step of fixation allows to improve the quality and the resolution of the labeling. The purpose of using a permeabilization, in particular a pre-extraction treatment with CSK buffer or PBS triton 0.5%, prior fixation is to remove soluble proteins and NA loosely bound to chromatin so that the only remaining staining is DNA bound compounds of the invention. By doing so, the resolution of the compounds of the invention cross-linked to DNA is higher due to a lower basal level of fluorescence. This enables the detection of foci targeted by compounds of the invention.

A method of the invention can in particular comprise at least one step of cell membrane permeabilization and at least one step of fixation between steps (b) and (c).

Moreover, the method may comprise at least one step of washing:
between steps (b) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, the method of the invention is such that it comprises:
at least one step of washing between steps (b) and (c); and
at least one step of washing between steps (c) and (d).

More particularly, the method of the invention can be such that it comprises:
between steps (b) and (c), at least one step of washing;
between steps (b) and (c), a step of cell membrane permeabilization followed by a step of fixation;

and
between steps (c) and (d), at least one step of washing; and even more particularly:
between steps (b) and (c), and in the following order, a step of washing, a step of cell membrane permeabilization, another step of washing, a step of fixation and another step of washing.

The step (d) of detecting the label in the cell obtained at step (c) may be performed by any detection method known by the person skilled in the art. In particular, the step (d) of detecting the label in the cell obtained at step (c) may be performed by microscopy or by flow cytometry.

Similarly, the present invention relates to the in vitro or ex vivo use of a compound of formula (I) according to the invention for visualizing DNA crosslinks in a cell, said compound of formula (I) according to the invention being used in combination with a label, preferably a fluorescent label, bearing a group complementary to the R¹ group of said compounds for a click-chemistry reaction.

The compound of Formula (I) according to the invention is thus useful to reveal ICL repair defects in a patient cells, in particular in patients selected from Fanconi Anemia (FA) patients, xeroderma pigmentosum (XP) patients, acute myeloid leukemia (AML) patients, head-and-neck cancer patients, ovarian cancer patients and breast cancer patients, more particularly in patients selected from Fanconi Anemia (FA) patients and xeroderma pigmentosum (XP) patients.

Alkylating agents are at the forefront of chemotherapy but their use is limited by their high toxicity for blood cells. Indeed, around ⅓ of patient will develop hematological complications. Such complications often result in dose reductions or treatment delays, which may compromise clinical outcome, or even mortality. Preventing discontinuation of treatment is therefore valuable for treating patients more effectively. One of the main causes of these side effects is the accumulation of ICL lesions that are not efficiently repaired. The compound of Formula (I) according to the invention is thus useful to evaluate the capacity of tumors for the repair of ICLs allowing to predict the sensitivity or resistance of a tumor to the treatment.

The present invention further relates to an in vitro or ex vivo method for detecting ICL repair defects in a patient cells, comprising at least the steps of:
(a) having at least one cell,
(b) contacting said cell with at least one compound of formula (I) according to the invention under conditions allowing said compound to induce DNA crosslinks in the cell;
(c) contacting the cell obtained in step (b) with at least one label, preferably a fluorescent label, bearing a group which is complementary for a click-chemistry reaction to the R¹ group of the compounds of formula (I) under conditions allowing the reaction of click-chemistry between the said R¹ group and the complementary group;
(d) measuring the labelling in the cell obtained at step (c); and
(e) optionally comparing the labeling measured at step (d) to a reference level.

In a particular embodiment, the patient is selected from Fanconi Anemia (FA) patients, xeroderma pigmentosum (XP) patients, acute myeloid leukemia (AML) patients, head-and-neck cancer patients, ovarian cancer patients and breast cancer patients, more particularly the patient is selected from Fanconi Anemia (FA) patients and xeroderma pigmentosum (XP) patients, even more particularly, the patient is a Fanconi Anemia (FA) patient.

In a particular embodiment, the cell is a cell which is resistant to an alkylating agent, in particular to a an alkylating agent selected from melphalan, chlorambucil, chlormethine and cyclophosphamide, more particularly to an alkylating agent selected from melphalan, chlorambucil and chlormethine, still more particularly to an alkylating agent selected from melphalan and chlorambucil, even more particularly to melphalan.

The resistance of a cell to an alkylating agent, in particular an alkylating agent selected from melphalan, chlorambucil, chlormethine and cyclophosphamide, more particularly to an alkylating agent selected from melphalan, chlorambucil and chlormethine, still more particularly to an alkylating agent selected from melphalan and chlorambucil, even more particularly to melphalan, refers to the incapacity of the agent to kill the cell, by apoptosis or any other killing process. Accordingly, the resistance of a cell to an alkylating agent, in particular to an alkylating agent selected from melphalan, chlorambucil, chlormethine and cyclophosphamide, more particularly to an alkylating agent selected from melphalan, chlorambucil and chlormethine, still more particularly to an alkylating agent selected from melphalan and chlorambucil, even more particularly to melphalan, is then inversely proportionate to the intensity of the label signal.

The reference level can be the intensity measured in a cell known for having a high or low resistance to an alkylating agent, in particular to an alkylating agent selected from melphalan, chlorambucil, chlormethine and cyclophosphamide, more particularly to an alkylating agent selected from melphalan, chlorambucil and chlormethine, still more particularly to an alkylating agent selected from melphalan and chlorambucil, even more particularly to melphalan. Preferably, the cell of reference is the closest of the cell to be studied. Alternatively, the reference level can be measured in a cell from the same patient, preferably a non-cancerous cell, for instance a corresponding histological normal reference tissue, in particular from the vicinity of the tumor.

The comparison of the labeling in optional step (e) can be used to detect ICL repair defects in a patient cells.

As defined herein above, a condition allowing a click-chemistry reaction, in particular a azide-alkyne cycloaddition (AAC) is the presence of copper. More particularly in said AAC, copper (I) act as a catalyst and can be provided by the use of copper (II) precursors with a reducing agent, such as sodium ascorbate or p-hydroquinone for instance, by copper (I) salts or by pre-formed copper (I) complexes.

In particular, the method can comprise a step of cell membrane permeabilization in particular a pre-extraction treatment with CSK buffer or PBS triton 0.5%, optionally followed by a step of fixation, between steps (b) and (c). The method can alternatively comprise a step of cell membrane permeabilization, optionally followed by a step of fixation, between steps (c) and (d). According to a particular embodiment, the method comprises a step of cell membrane permeabilization, optionally followed by a step of fixation, between steps (b) and (c) and a step of cell membrane permeabilization, optionally followed by a step of fixation, between steps (c) and (d).

A method of the invention can in particular comprise at least one step of cell membrane permeabilization in particular a pre-extraction treatment with CSK buffer or PBS triton 0.5%, and at least one step of fixation between steps (b) and (c).

Moreover, the method may comprise at least one step of washing:
between steps (b) and (c), in order to remove free compounds of the invention, i.e. compounds of the invention that are not bound to DNA of the cell; and/or
between steps (c) and (d), in order to remove free label as defined above, i.e. label that are not bound to compounds of the invention bound to DNA of the cell.

In a particular embodiment, the method of the invention is such that it comprises:
at least one step of washing between steps (b) and (c); and
at least one step of washing between steps (c) and (d).

More particularly, the method of the invention can be such that it comprises:
between steps (b) and (c), at least one step of washing;
between steps (b) and (c), a step of cell membrane permeabilization followed by a step of fixation;

and
between steps (c) and (d), at least one step of washing; and even more particularly:
between steps (b) and (c), and in the following order, a step of washing, a step of cell membrane permeabilization, another step of washing, a step of fixation and another step of washing.

The step (d) of measuring the labelling in the cell obtained at step (c) may be performed by any measuring method known by the person skilled in the art. In particular, the step (d) of detecting the label in the cell obtained at step (c) may be performed by microscopy or by flow cytometry.

In a particular embodiment, the cells used in the methods of the present invention are cancer cells. It can stem from a cancer cell line or a cell from primary tumors. It can be resistant to an alkylating agent, and in particular to an alkylating agent selected from melphalan, chlorambucil, chlormethine and cyclophosphamide, more particularly to melphalan. Preferably the cells are mammalian cells, and more specifically human cells.

The present invention also relates to an in vitro or ex vivo method for assessing the resistance or sensitivity of a tumor in a patient to a crosslinking agent, in particular to a compound of formula (I) as defined in any of claims 1 to 8, comprising at least the steps of:
(a) having at least one cell from the said tumor,
(b) contacting said cell with at least one said compound of formula (I) under conditions allowing said compound to induce DNA crosslinks in the cell,
(c) contacting the cell obtained in step (b) with at least one label bearing a group which is complementary for a click-chemistry reaction to the R¹ group of the compound of formula (I) under conditions allowing the reaction of click-chemistry between the said R¹ group and the complementary group,
(d) measuring the labeling in the cell obtained at step (c), and
(e) optionally comparing the labeling measured at step (d) to a reference level.

In a particular embodiment, the patient is selected from Fanconi Anemia (FA) patients, xeroderma pigmentosum (XP) patients, acute myeloid leukemia (AML) patients, head-and-neck cancer patients, ovarian cancer patients and breast cancer patients, more particularly the patient is selected from Fanconi Anemia (FA) patients and xeroderma pigmentosum (XP) patients, even more particularly, the patient is a Fanconi Anemia (FA) patient.

In all the methods of the invention, the labeling of DNA crosslinks authorizes the quantification of the number of DNA crosslinks generated by a compound of formula (I) of the invention. For example, if the label is fluorescent, the amount of fluorescence can be measured, this amount being proportional to the number of DNA crosslinks generated by a compound of formula (I) of the invention. If the label is radioactive, then the amount of radioactivity is measured.

All the methods of the invention may comprise at least one step of washing. The said at least one step of washing in a method of the invention may more particularly be performed after contacting a cell with a compound according to the invention and/or be performed after contacting a cell with a label as disclosed herein.

All the methods of the invention may comprise at least one step of cell membrane permeabilization. The said at least one step of cell membrane permeabilization (also termed cell permeabilization) in a method of the invention may more particularly be performed after contacting a cell with a compound according to the invention. The at least one step of permeabilization in a method of the invention can moreover be followed by a step of fixation, i.e. a step of fixation can be performed consecutively after the said at least one step of permeabilization.

In a particular embodiment, a method of the invention comprises at least one step of washing and at least one step of cell membrane permeabilization, the at least one step of cell membrane permeabilization being optionally followed by a step of fixation.

### Definitions

The definitions and explanations below are for the terms as used throughout the entire application, including both the specification and the claims.

Unless otherwise stated, any reference to compounds of the invention herein, means the compounds as such as well as their pharmaceutically acceptable salts and solvates.

When describing the compounds of the invention, the terms used are to be construed in accordance with the following definitions, unless indicated otherwise.

The term “halo” or “halogen” refers to the atoms of the group 17 of the periodic table (halogens) and includes in particular fluorine (F), chlorine (Cl), bromine (Br) and iodine (I) atom. Preferred halo gen atoms in the context of the invention are fluorine and chlorine, chlorine being particularly preferred.

The compounds of the invention containing a basic functional group may be in the form of pharmaceutically acceptable salts. Pharmaceutically acceptable salts of the compounds of the invention containing one or more basic functional groups include in particular the acid addition salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, cinnamate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Pharmaceutically acceptable salts of compounds of Formula I and subformulae may for example be prepared as follows:
(i) reacting the compound of Formula I or any of its subformulae with the desired acid; or
(ii) converting one salt of the compound of Formula I or any of its subformulae to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term “hydrate” is employed when said solvent is water.

The compounds of the invention include compounds of the invention as hereinbefore defined, including all polymorphs and crystal habits thereof, prodrugs and isomers thereof (including optical, geometric and tautomeric isomers) and isotopically-labeled compounds of the invention.

In addition, although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of the invention.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The term “therapeutically effective amount” (or more simply an “effective amount”) as used herein means the amount of active agent or active ingredient which is sufficient to achieve the desired therapeutic or prophylactic effect in the individual to which it is administered.

The term “administration”, or a variant thereof (e.g., “administering”), means providing the active agent or active ingredient, alone or as part of a pharmaceutically acceptable composition, to the patient in whom/which the condition, symptom, or disease is to be treated.

By “pharmaceutically acceptable” is meant that the ingredients of a pharmaceutical composition are compatible with each other and not deleterious to the patient thereof.

The term “excipient” as used herein means a substance formulated alongside the active agent or active ingredient in a pharmaceutical composition or medicament. Acceptable excipients for therapeutic use are well known in the pharmaceutical art, and are described, for example, in Remington's Pharmaceutical Sciences, 21^{st} Edition 2011. The choice of excipient can be selected with regard to the intended route of administration and standard pharmaceutical practice. The excipient must be acceptable in the sense of being not deleterious to the recipient thereof. The at least one pharmaceutically acceptable excipient may be for example, a binder, a diluent, a carrier, a lubricant, a disintegrator, a wetting agent, a dispersing agent, a suspending agent, and the like.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The present invention will be better understood with reference to the following examples and figures. These examples are intended to be representative of specific embodiments of the invention and are not intended as limiting the scope of the invention.

### FIGURES

FIG. 1: Modification for Click chemistry does not change the activities of click-melphalan or -monomelphalan. (A), (B) Hela cells exposed to various concentration of indicated drugs were subjected to clonogenic survival assays; (C) Representative image of comet assay conducted on Hela cells subjected to the indicated treatments; (D) Comet assay quantification indicating the decrease of the irradiation-induced comet tail moment with the indicated drugs; (E) Detection of FANCD2 and γ-H2AX in HeLa cell protein extracts after treatment with 1 μM melphalan or click-melphalan. Numbers under the blots indicate the fold induction relative to untreated samples. L/S indicates the ratio of monoubiquitinated (L, upper band) to non-monoubiquitinated (S, lower band) FANCD2; (F) Monitoring of cell cycle by Propidium iodide staining in Hela cells exposed to melphalan and click melphalan. Data are shown as mean±s.e.m. of 3 independent experiments, ns (not significant), *p<0.05, **p<0.01 and ****p<0.0001 according to two-way ANOVA followed by Šidák multiple range test (A, B) or one-way ANOVA non-parametric test (D) and Mann-Whitney test (E).

FIG. 2: (A) U2OS cells exposed to various concentrations of the indicated drugs were subjected to clonogenic survival assays; (B) IC50 of the indicated agents determined on HeLa Cells; (C) Comet assay quantification indicating the decrease of the irradiation-induced comet tail moment with the indicated drugs; (D) FANCD2, ERCC1, and GADPH detection in protein extracts from HeLa WT, FANCD2 KO and ERCC1 KO cell lines; (E) IC50 of the indicated agents determined on HeLa FANCD2 KO and ERCC1 KO cells; (F) HeLa WT, FANCD2 KO and ERCC1 KO exposed to several concentrations of click-melphalan were subjected to clonogenic survival assays; (G) Caspase 3/7 activation in response to high dose (50 μM) of melphalan or click-melphalan; (H) Detection of p53 expression by western blot in Hela cells 24 h after treatment with melphalan or click-melphalan. Data are shown as mean±s.e.m. of 3 independent experiments, ns (not significant), *p<0.05, **p<0.01 and ***p<0.001 according to two-way ANOVA followed by Šidák multiple range test (A) or by Tukey multiple range test (F) or to one-way ANOVA followed by Tukey's multiple comparison test (B, E, G) and Mann-Whitney test (C).

FIG. 3: Detection of click-melphalan and click-MonoMelphalan. (A) Schematic of click reaction between click-melphalan and Alexa fluor 647; (B) Detection of FANCD2 monoubiquitination with increased concentration of click-melphalan in Hela cells. L/S indicates the ratio of monoubiquitinated (L, upper band) to non-monoubiquitinated (S, lower band) FANCD2; (C) Representative images of click signals observed in Hela cells treated with the indicated drugs. The nucleus is stained in Dapi (blue) and agents are detected by clicking a Alexa Fluor 647 azide (red); (D) Detection of indicated agents by Flow cytometry after click reaction with Alexa Fluor 647.

FIG. 4: Repair of mono-melphalan and melphalan in WT and DNA repair deficient cells. (A) Quantification of HeLa cells positive for click-melphalan or click-mono-melphalan at the indicated time post treatment; (B) Same as A in HeLa and HeLa FANCD2 KO cells treated with click-melphalan; (C) Same as B with click-mono-melphalan; (D) Quantification of HeLa FANCD2 KO cells positive for click-mono-melphalan co-treated or not with non-clickable melphalan at the indicated time post treatment. Statistical analysis was performed in comparison with click-mono-melphalan 1 μM; (E) Quantification of HeLa cells positive for click-mono-melphalan co-treated or not with melphalan at the indicated time post treatment. Statistical analysis was performed in comparison with click-mono-melphalan 1 μM; (F) Quantification of click-melphalan and click-mono-melphalan positive cells at time 0 and 48 hours after treatment in BRCA2-deficient cells. Statistical analysis was performed in comparison with control cells. Positive cells are determined as cells with a fluorescence above background fluorescence (A-F). Data are shown as mean±s.e.m. of 3 independent experiments, ns (not significant), *p<0.05, **p<0.01, ***p<0.001 and ****p<0.0001 according to two-way ANOVA followed by Šidák multiple range test (A,-E) or one-way ANOVA followed by Dunn's multiple comparison test (F).

FIG. 5: (A) Jurkat or HeLa cells treated with click-mono-melphalan or click-melphalan were clicked with Alexa Fluor 647 at the indicated times post treatment and analyzed by flow cytometry; (B) Quantification of Jurkat cells positive for lesions induced by clickable molecules at the indicated time post treatment; (C) Quantification of fluorescence intensity in HeLa cells treated with click-melphalan or click-mono-melphalan at the indicated time post treatment; (D) Same as (C) in Jurkat cells. Data are shown as mean±s.e.m. of 3 independent experiments, ns (not significant), *p<0.05, **p<0.01 and ***p<0.001 according to two-way ANOVA followed by Šidák multiple range test.

FIG. 6: (A) HeLa WT and FANCD2 KO cells treated with click-mono-melphalan or click-melphalan were clicked with Alexa Fluor 647 at the indicated times post treatment and analyzed by flow cytometry; (B) Quantification of fluorescence intensity in WT and FANCD2 KO cell lines treated with click-melphalan at indicated times post treatment; (C)

Same as (B) with click-mono-melphalan treatment; (D) Same as (A) in RPE1 (control) cells and BRCA2-deficient cells; (E) Quantification of fluorescence intensity in BRCA2-KO cells treated with click-mono-melphalan; (F) Same as (E) with click-melphalan treatment. Data are shown as mean±s.e.m. of 3 independent experiments. *p<0.05, ***p<0.001 and ***p<0.0001 according to two-way ANOVA followed by Šidák multiple range test.

FIG. 7: Analysis of the FA-dependent melphalan-ICL repair during the cell cycle. (A) Dual parameter plot of the click-mono-melphalan or melphalan subjected to the Alexa 647-click reaction with propidium iodide co-staining. Lesions repair is evaluated in HeLa and HeLa FANCD2 KO cells by flow cytometry at different time post treatment. The percentage of G1 phase cells is indicated in the left corner (black), S phase cells in the center (yellow) and G2 phase in the right corner (red); (B) HeLa cells were arrested in G1 phase using a double thymidine block. Cultures harvested at various time points after release were stained with propidium iodide and analyzed using flow cytometry; (C) Quantification of fluorescence intensity in HeLa WT or HeLa FANCD2 KO treated with click-melphalan clicked with Alexa Fluor 647 azide at the indicated time post treatment. Data are shown as mean±s.e.m. of 3 independent experiments. *p<0.05 according to two-way ANOVA followed by Šidák multiple range test.

FIG. 8: (A) HeLa WT and FANCD2 KO cells were synchronized at the G1/S boundary, treated or not with click-melphalan before release into drug free medium. Cell cycle profile was monitored by propidium iodide staining; (B) Quantification of fluorescence intensity in HeLa WT and FANCD2 KO cell lines treated with click-melphalan at indicated times post treatment.

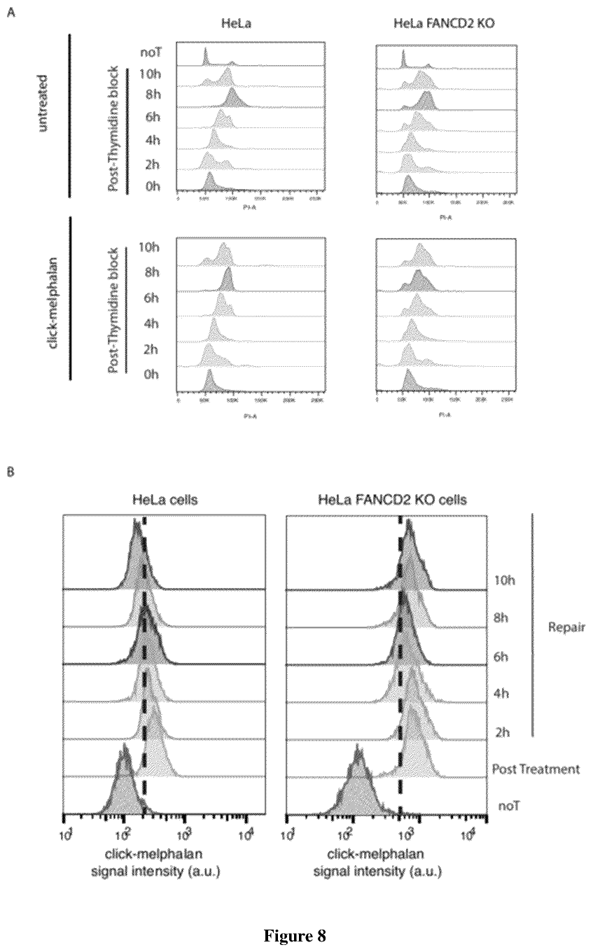

FIG. 9: Diagnostic of FA using click-melphalan. (A) Table recapitulating the cellular and molecular phenotypes of cells from potential FA patients (FA 1 and FA 2) or healthy subjects (Control 3 and Control 4); (B) Patient cells treated with click-mono-melphalan are clicked with Alexa Fluor 647 at the indicated times post treatment and analyzed by flow cytometry; (C) Quantification of patient cells positive for click-mono-melphalan at the indicated time post treatment; (D) Patient cells treated with click-melphalan labeled with Alexa Fluor 647 at the indicated times post treatment and analyzed by flow cytometry; (E) Quantification of patient cells positive for click-melphalan at the indicated time post treatment. Data are shown as mean±s.e.m. of 3 independent experiments, ns (not significant), *p<0.05, ****p<0.0001 according to two-way ANOVA followed by Tukey's multiple range test.

FIG. 10: (A) FANCD2 ubiquitination in protein extracts from the two primary FA patient fibroblasts (FA1 and FA2) and controls; (B) FANCD2, p53, γH1AX and GAPDH immunoblotting in protein extracts from FA1 and FA, and from healthy fibroblasts C3 and C4; (C) Flow cytometry-based MMC intensity test in primary FA fibroblast from FA1 and FA2, compared to a FA and a non-FA controls. The fraction of dying cells is marked in black. The number of dying cells increases in FA after 1 ng/ml of MMC and in control cells after 10 ng/ml of MMC (pointed with arrows).

FIG. 11: Diagnostic of Xeroderma pigmentosum (XP) patients based on click-melphalan and click-mono-melphalan detection. (A) Patient cells treated with click-mono-melphalan are clicked with Alexa Fluor 647 at the indicated times post treatment and analyzed by flow cytometry; (B) Quantification of patient cells positive for click-mono-melphalan at the indicated time post treatment; (C) Patient cells treated with click-melphalan labeled with Alexa Fluor 647 at the indicated times post treatment and analyzed by flow cytometry; (D) Quantification of patient cells positive for click-melphalan at the indicated time post treatment. Data are shown as mean±s.e.m. of 3 independent experiments. *p<0.05 **p<0.01 according to two-way ANOVA followed by Tukey's multiple range test.

FIG. 12: (A) Patient PHA-stimulated lymphocytes treated with click-mono-melphalan were clicked with Alexa Fluor 647 post treatment or after 48 h and analyzed by flow cytometry; (B) Quantification of patient cells positive for click-mono-melphalan; (C) Patient cells treated with click-melphalan labeled with Alexa Fluor 647 post treatment or after 48 h and analyzed by flow cytometry; (D) Quantification of patient cells positive for click-melphalan. Data are shown as mean±s.e.m. of 3 independent experiments. ****p<0.0001 according to two-way ANOVA followed by Šidák multiple range test.

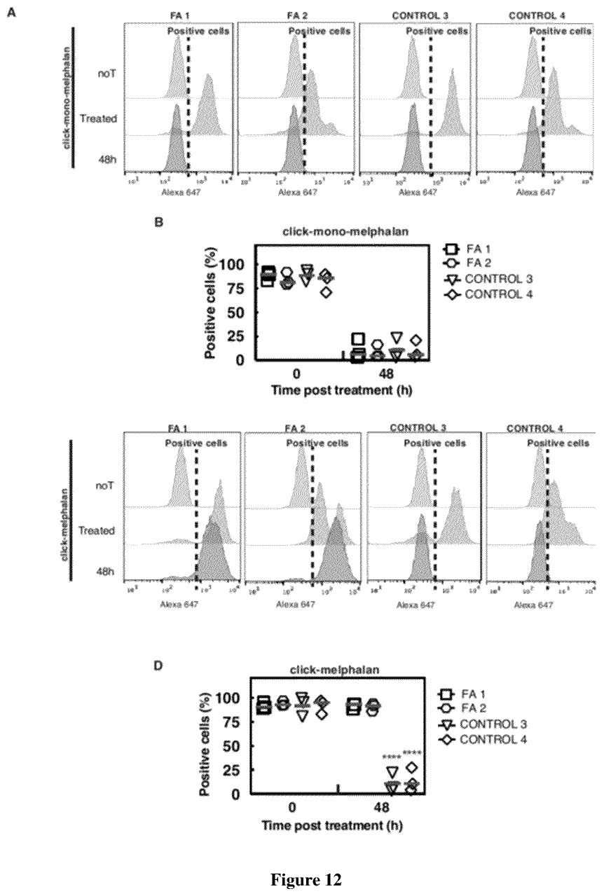

### EXAMPLES

### Abbreviations

CSK: cytoskeletal;
DAD: diode-array detection;
DAPI: 4′,6-diamidino-2-phenylindole;
DCM: dichloromethane;
DMSO: dimethylsulfoxide;
DMEM: Dulbecco's modified Eagle's medium;
DNA: deoxyribonucleic acid;
ECL: enhanced chemiluminescence;
EDTA: ethylenediaminetetraacetic acid;
ESI: electrospray ionization;
FA: Fanconi anemia;
FBS: fetal bovine serum;
FCS: fetal calf serum;
HEPES: N-2-hydroxyethylpiperazine-N′-2-ethanesulfonic acid;
HPLC: high-performance liquid chromatography;
HRP: horseradish peroxide;
ICL: interstrand crosslink;
IR: infrared;
LC-MS: liquid chromatography-mass spectrometry;
MEM: Modified Eagle Medium;
MMC: Mitomycin C;
MS: mass spectrometry;
NER: Nucleotide excision repair;
NM: nitrogen mustard;
NMR: nuclear magnetic resonance;
PBLs: peripheral blood lymphocytes;
PBS: phosphate-buffered saline;
PHA: phytohemagglutinin;
PI: propidium iodide;
PIPES: piperazine-N,N′-bis(2-ethanesulfonic acid;
PMSF: phenylmethanesulfonyl fluoride;
ppm: parts per million;
RIPA: radioimmunoprecipitation assay;
RPMI: Roswell Park Memorial Institute medium;
Rt: retention time;
SD: standard deviation;
SDS: sodium dodecyl sulfate;
SYBR green I: N′,N′-dimethyl-N-[4-[(E)-(3-methyl-1,3-benzothiazol-2-ylidene)methyl]-1-phenylquinolin-1-ium-2-yl]-N-propylpropane-1,3-diamine;
TLC: thin-layer chromatography;
UV: ultraviolet;
XP: Xeroderma pigmentosum.

### Chemistry

### General Methods

Commercially available reagents and solvents were used without further additional purification. Thin layer chromatography (TLC) was performed on precoated aluminum sheets of silica (60 F₂₅₄ nm, Merck) and visualized using short-wave UV light. Reaction monitoring and purity of compounds were recorded by using analytical Agilent Infinity high performance liquid chromatography with DAD at 254 nM (Column Zorbax SB-C18 1.8 UM (2.1×50 mm), mobile phase (A: 0.1% FA H₂O, B: 0.1% FA MeCN), flow rate 0.3 mL/min, time/% B 0/10, 4/90, 7/90, 9/10, 10/10. Column chromatography was performed on a Reveleris purification system using Reveleris Flash silica cartridges. Preparative high-performance liquid chromatography (HPLC) was carried out using an Agilent 1290 Infinity II instrument with DAD at 254 nM (Column Thermo Scientific ODS Hypersil 10 μM (10×250 mm), mobile phase (A: 0.1% TFA H₂O, B: 0.1% TFA MeCN), flow rate 5 mL/min, time/% B 0/10, 8/90, 12/90, 13/10, 16/10. Petroleum refers to the fraction with distillation range 40-65° C. ¹H and ¹³C NMR spectra were recorded by using a Bruker AC 400 spectrometer. Chemical shifts, (8) are reported in ppm and coupling values (J) in hertz. Abbreviations for peaks are, br: broad, s: singlet, d: doublet, t: triplet, q: quadruplet, quint: quintuplet, sex: sextuplet and m: multiplet). The spectra recorded are consistent with the proposed structures. Low-resolution mass spectra were obtained with Agilent SQ G6120B mass spectrometer in positive and negative electrospray mode. High-resolution mass spectra were performed at the Spectropole Analytical Laboratory of the University Aix-Marseille.

### Synthesis

### Example 1: (S)-2-Amino-3-(4-(bis(2-chloroethyl)amino)phenyl)-n-(prop-2-yn-1-yl)propenamide (4, Click-Melphalan)

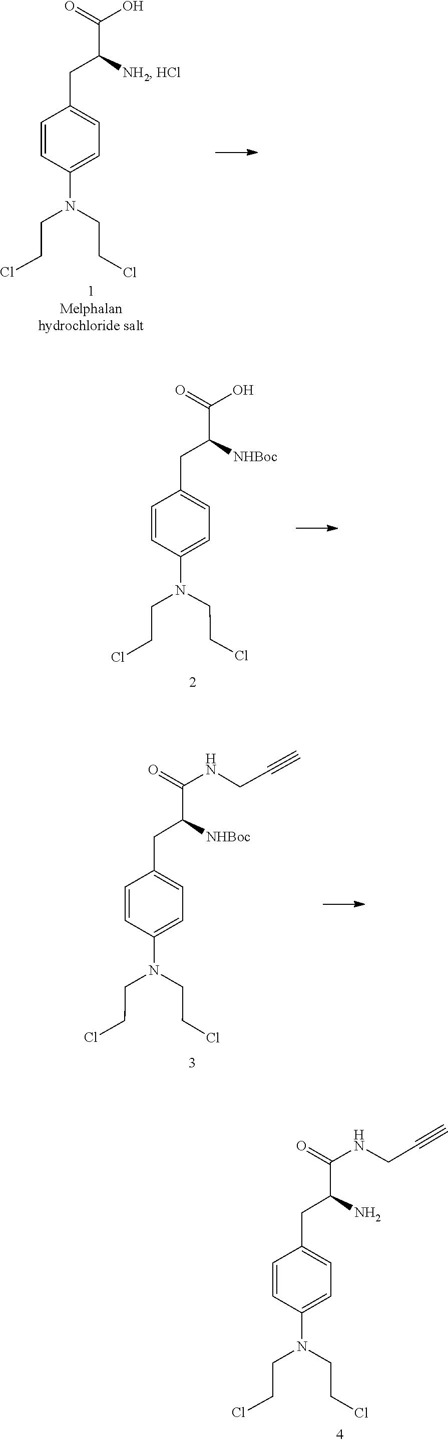

(S)-3-(4-(bis(2-Chloroethyl)amino)phenyl)-2-((tert-butoxycarbonyl)amino)propanoic acid (2). At 0° C., to a solution of(S)-2-amino-3-(4-(bis(2-chloroethyl)amino)phenyl)propanoic acid hydrochloride salt (1) (840 mg, 2.46 mmol) in methanol (60 mL) were added triethylamine (1.37 mL, 9.84 mmol) and di-tert-butyl dicarbonate (1.13 mL, 4.92 mmol) successively. The resulting mixture was allowed to warm to room temperature and stirred for a further 30 min. The solvent was evaporated under reduced pressure and the residue was taken up in 0.01 N HCl aqueous solution (50 mL) and extracted with AcOEt (3×30 mL). The combined organic layers were washed with brine and dried over Na₂SO₄. The solvent was evaporated under reduced pressure to afford (S)-3-(4-(bis(2-chloroethyl)amino)phenyl)-2-((tert-butoxycarbonyl)amino) propanoic acid (2) as a light yellow oil which crystallised under air (1.75 g, >100%), and which was used in the following step without further purification.

LC-MS C₁₈H₂₆C₁₂N₂O₄: Rt 8.256 min, ESI(+) 405.2 [M+H]⁺.

¹H NMR (400 MHz, CDCl₃) δ 7.06 (d, J=7.9 Hz, 2H), 6.57 (d, J=7.9 Hz, 2H), 5.25-5.11 (m, 1H), 4.43-4.32 (m, 1H), 3.71-3.56 (m, 8H), 3.07-2.85 (m, 2H) and 1.37 (s, 9H) ppm.

tert-Butyl-(S)-(3-(4-(bis(2-chloroethyl)amino)phenyl)-1-oxo-1-(prop-2-yn-1-ylamino)propan-2-yl) carbamate (3). To a solution of (2) (997 mg, 2.46 mmol) in N,N-dimethylformamide (6 mL) was added a solution of (benzotriazol-1-yloxy)tris(dimethylamino)phosphonium hexafluorophosphate (1.09 g, 2.46 mmol) in N,N-dimethylformamide (3 mL). After stirring at room temperature for 10 min, a solution of propargylamine (0.157 mL, 2.46 mmol) in N,N-dimethylformamide (3 mL) was injected slowly, dropwise. After addition, the solution was stirred for a further 15 min, then triethylamine (685 μL, 4.92 mmol) was added dropwise. The resulting mixture was stirred at room temperature overnight, then concentrated under reduced pressure. The residue was purified by flash chromatography, using a DCM/MeOH gradient (100:0 to 98:2), to afford tert-butyl-(S)-(3-(4-(bis(2-chloroethyl)amino)phenyl)-1-oxo-1-(prop-2-yn-1-ylamino)propan-2-yl) carbamate (3) as a light yellow oil (970 mg, 89%).

LC-MS C₂₁H₂₉C₁₂N₃O₃: Rt 8.257 min, ESI(+) 442.1 [M+H]⁺.

¹H NMR (400 MHz, CDCl₃) δ 7.09 (d, J=8.6 Hz, 2H), 6.66 (d, J=8.6 Hz, 2H), 6.06 (t, J=5.2 Hz, 1H), 5.13-4.91 (m, 1H), 4.26 (q, J=6.7 Hz, 1H), 3.99 (dd, J=5.2, 2.5 Hz, 2H), 3.78-3.53 (m, 8H), 2.96 (d, J=6.7 Hz, 2H), 2.20 (t, J=2.5 Hz, 1H), 1.41 (s, 9H) ppm.

(S)-2-Amino-3-(4-(bis(2-chloroethyl)amino)phenyl)-N-(prop-2-yn-1-yl)propanamide (4). To a solution of (3) (970 mg, 2.20 mmol) in diethyl ether (100 mL) was added a 2 N hydrochloric acid in diethyl ether (10 mL, 20 mmol). After addition, the stirring was maintained for 4 h. The resulting mixture was extracted with ice cold water (4×20 mL) and combined aqueous layers were washed twice with Et₂O (2×20 mL). The aqueous layer was basified with a saturated Na₂CO₃ aqueous solution until pH=8, then extracted with DCM (3× 20 mL). The solvent was evaporated under reduced pressure and the residue was purified by flash chromatography, using a DCM/MeOH gradient (100:0 to 95:5), to afford (S)-2-amino-3-(4-(bis(2-chloroethyl)amino)phenyl)-N-(prop-2-yn-1-yl)propanamide (4) as a yellow oil which crystallised from a CHCl₃/Et₂O mixture as a white powder (708 mg, 94%).

LC-MS C₁₆H₂₁Cl₂N₃O: Rt 6.579 min, ESI(+) 342.1 [M+H]⁺.

HRMS ESI(+) calcd 342.1134 [M+H]⁺, found 342.1132.

¹H NMR (400 MHz, MeOD) δ 7.08 (d, J=8.7 Hz, 2H), 6.69 (d, J=8.7 Hz, 2H), 3.97 (dd, J=17.5, 2.5 Hz, 1H), 3.92 (dd, J=17.5, 2.5 Hz, 1H), 3.77-3.63 (m, 8H), 3.46 (t, J=6.7 Hz, 1H), 2.87 (dd, J=13.5, 6.7 Hz, 1H), 2.72 (dd, J=13.5, 6.7 Hz, 1H), 2.57 (t, J=2.5 Hz, 1H) ppm.

¹³C NMR (101 MHz, MeOD) δ 176.56, 146.55, 131.59, 127.07, 113.47, 80.44, 72.21, 57.68, 54.43, 49.00, 41.66, 29.32 ppm.

### Comparative Example 1: (S)-2-Amino-3-(4-((2-chloroethyl) (2-hydroxyethyl)amino)phenyl)propanoic acid (5, Mono-Melphalan)

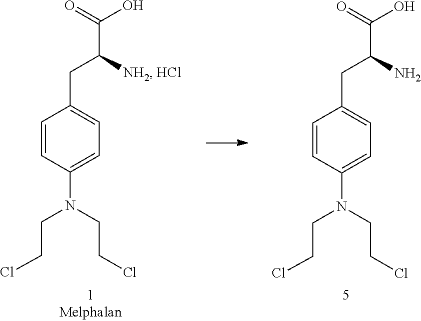

(S)-2-Amino-3-(4-((2-chloroethyl) (2-hydroxyethyl)amino)phenyl)propanoic acid (5). A solution of (1) (153 mg, 0.5 mmol) in a mixture of 1 N hydrochloric acid aqueous solution (2 mL) and dimethylsulfoxyde (1 mL) was heated at 90° C. for 50 min. The reaction was monitored by LC-MS until completion. The resulting mixture was purified without any work up by HPLC to afford (S)-2-amino-3-(4-((2-chloroethyl)(2-hydroxyethyl)amino)phenyl)propanoic acid (5) as a white powder (65 mg, 45%).

LC-MS C₁₃H₁₉ClN₂O₃: Rt 5.404 min, ESI(+) 287.1 [M+H]⁺.

HRMS ESI(+) calcd 309.0976 [M+Na]+, found 309.0976.

¹H NMR (400 MHz, MeOD) δ 7.13 (d, J=8.8 Hz, 2H), 6.77 (d, J=8.8 Hz, 2H), 4.15 (dd, J=7.7, 5.2 Hz, 1H), 3.76-3.72 (m, 2H), 3.70-3.64 (m, 4H), 3.55 (t, J=6.1 Hz, 2H), 3.20 (dd, J=14.6, 5.2 Hz, 1H), 3.04 (dd, J=14.6, 7.7 Hz, 1H) ppm.

¹³C NMR (101 MHz, MeOD) δ 171.41, 147.96, 131.53, 123.18, 113.86, 60.08, 55.33, 54.63, 54.54, 41.17, 36.39 ppm.

### Example 2: (S)-2-Amino-3-(4-((2-chloroethyl)(2-hydroxyethyl)amino)phenyl)-n-(prop-2-yn-1-yl)propenamide (6, Click-Mono-Melphalan)

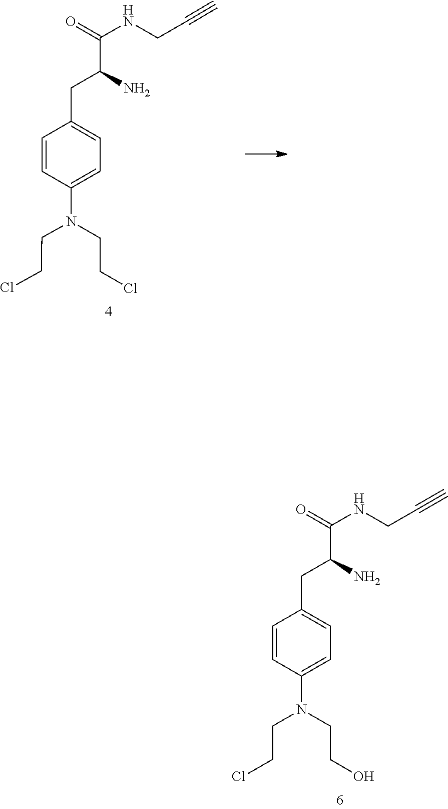 [SMILES: C#CCNC(=O)[C@@H](N)Cc1ccc(N(CCCl)CCCl)cc1 ; C#CCNC(=O)[C@@H](N)Cc1ccc(N(CCO)CCCl)cc1]

(S)-2-Amino-3-(4-((2-chloroethyl)(2-hydroxyethyl)amino)phenyl)-N-(prop-2-yn-1-yl)propanamide (6). A solution of (4) (337 mg, 1 mmol) in a mixture of 1 N hydrochloric acid aqueous solution (2 mL) and dimethylsulfoxyde (1 mL) was heated at 90° C. for 1 h 30. The reaction was monitored by LC-MS until completion. The resulting mixture was purified without any work up by HPLC to afford (S)-2-amino-3-(4-((2-chloroethyl)(2-hydroxyethyl)amino)phenyl)-N-(prop-2-yn-1-yl)propanamide (6) as a white powder (130 mg, 41%).

LC-MS C₁₆H₂₂ClN₃O₂: Rt 5.737 min, ESI(+) 324.2 [M+H]⁺.

HRMS ESI(+) calcd 324.1473 [M+H]⁺, found 324.1473.

¹H NMR (400 MHz, MeOD) δ 7.11 (d, J=8.7 Hz, 2H), 6.76 (d, J=8.7 Hz, 2H), 4.01 (dd, J=17.5, 2.5 Hz, 1H), 3.96 (dd, J=17.5, 2.5 Hz, 1H), 3.95 (t, J=7.2 Hz, 1H), 3.74 (t, J=6.4 Hz, 2H), 3.70-3.63 (m, 4H), 3.54 (t, J=6.0 Hz, 2H), 3.07 (dd, J=14.1, 7.2 Hz, 1H), 2.95 (dd, J=14.1, 7.2 Hz, 1H), 2.65 (t, J=2.5 Hz, 1H) ppm.

¹³C NMR (101 MHz, MeOD) δ 169.44, 147.81, 131.58, 123.23, 113.87, 79.83, 72.79, 60.04, 55.91, 54.68, 54.57, 41.16, 37.66, 29.57 ppm.

### Example 3: (S)-2-Amino-3-(4-(bis(2-chloroethyl)amino)phenyl)-n-(pent-4-yn-1-yl)propanamide (8)

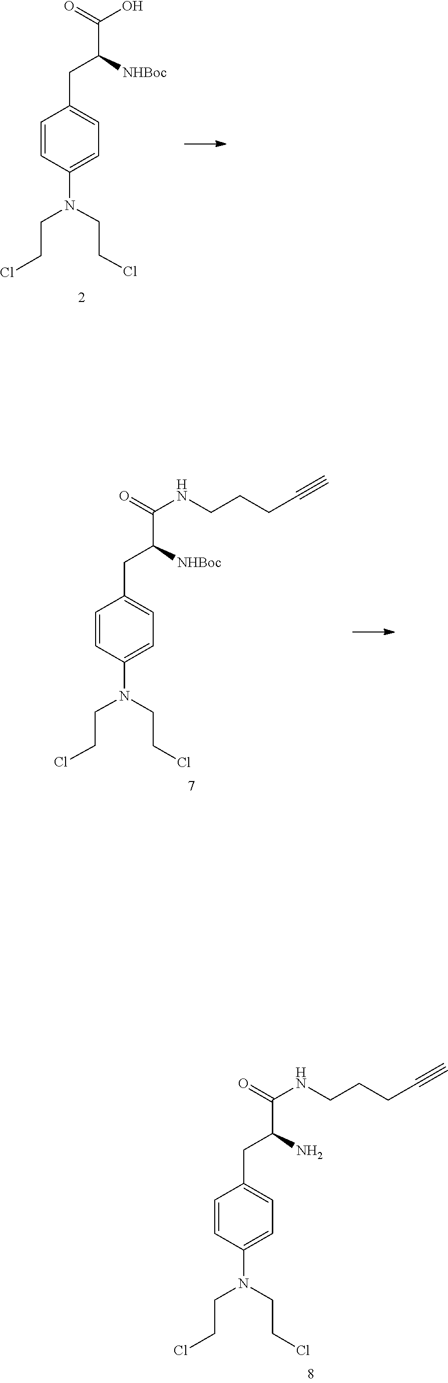 [SMILES: CC(C)(C)OC(=O)N[C@@H](Cc1ccc(N(CCCl)CCCl)cc1)C(=O)O ; C#CCCCNC(=O)[C@H](Cc1ccc(N(CCCl)CCCl)cc1)NC(=O)OC(C)(C)C ; C#CCCCNC(=O)[C@@H](N)Cc1ccc(N(CCCl)CCCl)cc1]

tert-Butyl(S)-(3-(4-(bis(2-chloroethyl)amino)phenyl)-1-oxo-1-(pent-4-yn-1-ylamino)propan-2-yl)carbamate (7). Using the same procedure as described for compound (3) to afford tert-butyl(S)-(3-(4-(bis(2-chloroethyl)amino)phenyl)-1-oxo-1-(pent-4-yn-1-ylamino)propan-2-yl)carbamate (7) as a light yellow solid (1.06 g, 92%).

LC-MS C₂₃H₃₃Cl₂N₃O₃: Rt 8.568 min, ESI(+) 470.9 [M+H]⁺.

¹H NMR (400 MHz, CDCl₃) δ 7.06 (d, J=8.6 Hz, 2H), 6.60 (d, J=8.6 Hz, 2H), 5.89 (sbr, 1H), 5.00 (sbr, 1H), 4.19 (q, J=6.7 Hz, 1H), 3.71-3.58 (m, 8H), 3.33-3.19 (m, 2H), 2.98 (dd, J=13.5, 6.7 Hz, 1H), 2.86 (dd, J=13.5, 6.7 Hz, 1H), 2.21 (td, J=5.5, 2.7 Hz, 1H), 2.05-2.10 (m, 2H), 1.62-1.57 (m, 2H), 1.40 (s, 9H) ppm.

(S)-2-Amino-3-(4-(bis(2-chloroethyl)amino)phenyl)-N-(pent-4-yn-1-yl)propanamide (8). Using the same procedure as described for compound (4) to afford (S)-2-amino-3-(4-(bis(2-chloroethyl)amino)phenyl)-N-(pent-4-yn-1-yl)propanamide (8) as a yellow powder (707 mg, 87%).

LC-MS C₁₈H₂₅Cl₂N₃O: Rt 7.311 min, ESI(+) 370.9 [M+H]⁺.

¹H NMR (400 MHz, MeOD) δ 7.07 (d, J=8.7 Hz, 2H), 6.70 (d, J=8.7 Hz, 2H), 3.75-3.72 (m, 4H), 3.67-3.64 (m, 4H), 3.44 (t, J=6.7 Hz, 1H), 3.27 (dd, J=13.5, 6.7 Hz, 1H), 3.12 (dd, J=13.5, 6.7 Hz, 1H), 2.85-2.71 (m, 2H), 2.23 (t, J=2.7 Hz, 1H), 2.04-1.93 (m, 2H), 1.56 (quint, J=7.0 Hz, 2H) ppm.

¹³C NMR (101 MHz, MeOD) δ 176.66, 146.57, 131.53, 127.12, 113.37, 84.30, 70.06, 57.95, 54.42, 41.67, 41.62, 39.21, 29.28, 16.54 ppm.

### Example 4: (S)-2-Amino-N-(3-azidopropyl)-3-(4-(bis(2-chloroethyl)amino)phenyl)propanamide (10)

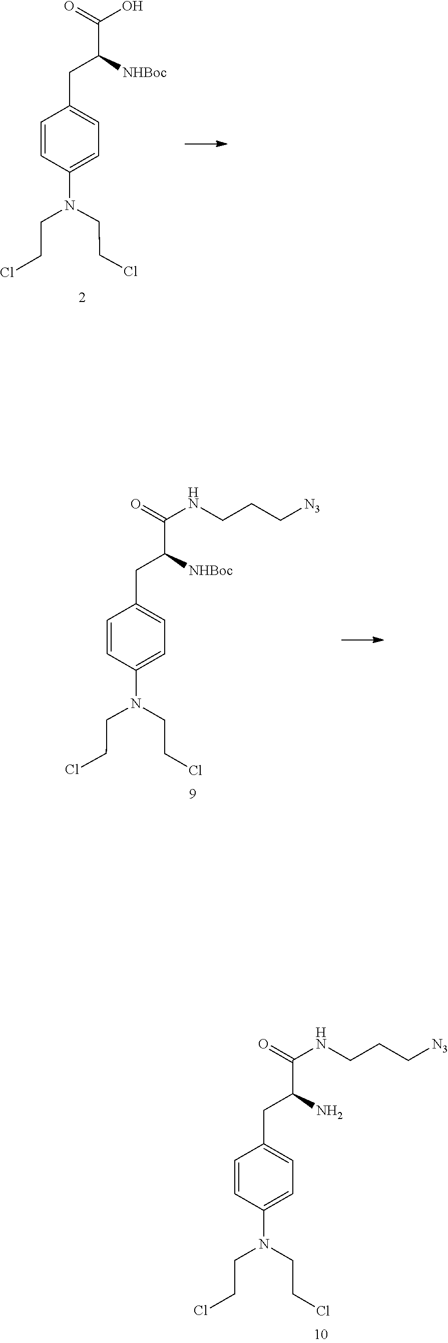 [SMILES: CC(C)(C)OC(=O)N[C@@H](Cc1ccc(N(CCCl)CCCl)cc1)C(=O)O ; CC(C)(C)OC(=O)N[C@@H](Cc1ccc(N(CCCl)CCCl)cc1)C(=O)NCCCN=[N+]=[N-] ; [N-]=[N+]=NCCCNC(=O)[C@@H](N)Cc1ccc(N(CCCl)CCCl)cc1]

(S)-tert-Butyl (1-((3-azidopropyl)amino)-3-(4-(bis(2-chloroethyl)amino)phenyl)-1-oxopropan-2-yl)carbamate (9). Using the same procedure as described for compound (3) to afford (S)-tert-butyl (1-((3-azidopropyl)amino)-3-(4-(bis(2-chloroethyl)amino)phenyl)-1-oxopropan-2-yl)carbamate (9) as a light orange oil (593 mg, 50%).

LC-MS C₂₁H₃₂Cl₂N₆O₃: Rt 8.634 min, ESI(+) 487.8 [M+H]⁺.

¹H NMR (400 MHz, CDCl₃) δ 7.08 (d, J=8.6 Hz, 2H), 6.62 (d, J=8.6 Hz, 2H), 5.95 (sbr, 1H), 5.01 (sbr, 1H), 4.19 (q, J=6.7 Hz, 1H), 3.77-3.66 (m, 4H), 3.66-3.52 (m, 4H), 3.35-3.15 (m, 4H), 2.99 (dd, J=13.7, 6.7 Hz, 1H), 2.89 (dd, J=13.7, 6.7 Hz, 1H), 1.57 (quint, J=6.6 Hz, 2H), 1.42 (s, 9H) ppm.

(S)-2-Amino-N-(3-azidopropyl)-3-(4-(bis(2-chloroethyl)amino)phenyl)propanamide (10). Using the same procedure as described for compound (4) to afford (S)-2-amino-N-(3-azidopropyl)-3-(4-(bis(2-chloroethyl)amino)phenyl)propanamide (10) as a yellow powder (627 mg, 74%).

LC-MS C₁₆H₂₄Cl₂N₆O: Rt 7.358 min, ESI(+) 387.9 [M+H]⁺.

¹H NMR (400 MHz, MeOD) δ 7.07 (d, J=8.8 Hz, 2H), 6.70 (d, J=8.8 Hz, 2H), 3.74 (m, 4H), 3.70-3.60 (m, 4H), 3.44 (t, J=6.8 Hz, 1H), 3.25 (dd, J=13.5, 6.8 Hz, 1H), 3.18-3.02 (m, 3H), 2.87-2.70 (m, 2H), 1.60 (quint, J=6.6 Hz, 2H) ppm.

¹³C NMR (101 MHz, MeOD) δ 176.81, 146.63, 131.56, 127.19, 113.41, 58.03, 54.43, 49.79, 41.67, 37.37, 29.52 ppm.

### Biological Evaluation

### Materials and Methods

### 1. Cell Culture

HeLa, HeLa FANCD2 KO cells were maintained at sub-confluent levels in Dulbecco's modified Eagle's medium (DMEM; Gibco) supplemented with 10% fetal bovine serum (Thermo Scientific, Waltham, MD) at 37° C. in a humidified atmosphere containing 5% CO₂. Mononuclear peripheral blood cells were purified from fresh blood samples using Ficoll-Paque Plus (Amersham Biosciences, Saclay, France). 10×10⁶ cells were cultured at a concentration of 106/mL in RPMI-FBS (RPMI 1640, 10% vol/vol fetal bovine serum [FBS]), and T-cells were stimulated with phytohemagglutinin (PHA) for 72 hours without exposure to any DNA-damaging agent. For primary fibroblasts culture, 4-mm-square skin biopsies were performed using local anesthesia with Emla (Astra, Rueil, France) and the Biopsy Punch (Stiefel, Rueil, France). Skin samples were thinly sliced, and the resulting fragments were adhered on plastic plates for 15 minutes then covered with modified Eagle medium (MEM) supplemented with antibiotic, HEPES (N-2-hydroxyethylpiperazine-N′-2-ethanesulfonic acid), MEM nonessential amino acids (Invitrogen, Cergy Pontoise, France), and 20% fetal calf serum (FCS) (GIBCO-BRL Life Technologies, Cergy Pontoise, France), at 37° C. with 5% CO₂. Individual growing clones were picked up after 2 to 3 weeks and expanded. Cells were grown and tested without exposure to DNA damaging agent.

### 2. Clonogenic Survival Analysis in Hela Cells

1000 Hela cells were seeded in triplicate in 10 cm dishes and allowed to attach for 4 hours before treatment for 1 hour with the indicated drugs. After 15 days, cells were washed, fixed and stained with crystal violet (Sigma). The number of colonies with >100 cells were counted. Results were normalized to plating efficiency. For each genotype, cell viability of untreated cells was defined as 100%. Data are represented as mean±SD from three independent experiments.

### 3. Detection of G2 Arrest by Flow Cytometry

Low-confluence cultures of asynchronously growing Hela cells were treated with the indicated drugs at the indicated concentration for 1 h and left for 24 hours in culture media. Following trypsinization, cells were collected by centrifugation, washed in PBS, and fixed in ice-cold 70% ethanol. Cells were centrifuged, washed in PBS, and treated for 15 min at 37° C. with 50 μg/mL RNase A. Cells were stained at room temperature with propidium iodide (PI) solution (2 mM MgCl₂, 10 mM PIPES buffer, 0.1 M NaCl, 0.1% Triton X-100, 0.01 mg/mL PI) and analyzed on a LSR Fortessa flow cytometer (BD Biosciences, San Jose, CA). Cell cycle profiles were created using FlowJo analysis software (Tree Star, Inc.).

### 4. Measurement of Inhibitory Concentration 50 Values

In antiproliferative assays, compounds were assayed for their growth inhibiting activity towards the described cancer cell lines using the CellTiter-Glo Luminesent Cell Viability Assay as described by the manufacturer (Promega Corporation). Briefly, 5.10³ cells were plated onto 96 well-plates (white with clear bottom (3610, Corning Costar) with 90 μL media per well, and incubated overnight. Compounds were added at different concentrations (varying from 50 to 0.032 μM) to each well and cell cultures were incubated at 37° C. during 72 h. Vehicle (DMSO) was used as control and all compounds were tested in constant percentage of DMSO (0.5%). After addition of 50 μL CellTiter GLO, luminescence was measured using a Centro luminometer LB960 (Berthold). Dose-response curves were generated and effective dose 50 values (EC50) were calculated using nonlinear regression analysis (Graph Pad Prism).

### 5. Reverse Alkaline Comet Assay

HeLa cells (100,000 per well) were seeded into 6-well cluster plates and allowed to settle overnight. Cells were initially treated with DMSO vehicle, 30 μM Melphalan or 30 μM Click-Melphalan (Example 1) for 60 min and then thoroughly washed with PBS. Following treatment, cell samples were treated with PBS as a control or IR (10 Gy) before harvest by trypsinization. Detached samples were subsequently resuspended in molten 1% Type VII low gelling temperature agarose and then allowed to set on glass slides pre-coated with agarose. Cells were then lysed by bathing slides in ice-cold lysis buffer (100 mM Na₂EDTA, 2.5 M NaCl, 10 mM Tris-HCl (pH 10.5), 1% Triton X-100) for 60 min and then subjected to 4×15 min washes with ice-cold MilliQ H₂O. Each slide was then submerged in alkali electrophoresis buffer (300 mM NaOH, 1 mM Na₂EDTA) for 60 min and then electrophoresed at 30 V for 30 min at 4° C. Samples were neutralized by the addition of neutralization buffer (500 mM Tris-HCl pH 7.5) for 10 min and then allowed to dry overnight at ambient temperature. Comets were stained with SYBR green I for 10 min and then washed using 3×MilliQ H₂O washes. Samples were visualized using a Zeiss AXIO 2 with a 20×/0.5 NA Plan Apo objective and the level of DNA damage assessed using OpenComet. At least 50 comets were scored per slide.

### 6. Detection of FANCD2 Ubiquitination, γH2Ax and p53

Low-confluence cultures of asynchronously growing HeLa cells or primary FA fibroblasts were treated with the drugs at the indicated concentration for 1 h and left for 24 hours in media culture for the detection of FANCD2 and γH2AX. HeLa cells were treated with 1 μM of indicated drugs for 24 hours in media culture for the detection of p53. Following trypsinization, cells were watched in PBS and lysed in RIPA buffer (50 mM Tris-HCl at pH 7.5, 150 mM NaCl, 0.5% sodium deoxycholate, 0.1% SDS, 1% NP-40, 5 mM EDTA, 20 mM β-glycerophosphate, 50 mM NaF, 1× protease inhibitor (Complete EDTA-free tablet Roche), 50 U/mL Benzonase, 1 mM PMSF). Samples were separated by SDS-polyacrylamide gel electrophoresis, transferred to a membrane, and detected with anti FancD2 (abcam ab108928), anti γH2Ax (cell signaling #9718) or anti p53 (Santa Cruz Biotechnology sc-126) and ECL reagents (GE Healthcare) using chemidoc (Biorad).

### 7. Caspase Assay

Caspase 3/7 activation was analyzed in HeLa cells treated with the indicated compounds for 16 hours using the Promega Caspase Glo assay. Briefly, 5×10³ cells were plated onto 96-well plates in 100 μL of media per well 6 hours before the assay. Compounds were added at 50 μM to each well, and cell cultures were incubated at 37° C. for 16 hours. Vehicle (DMSO) was used as a control, and all compounds were tested at a constant percentage of DMSO (0.5%). After adding 50 μL of Caspase 3/7 GLO, luminescence was measured using a Centro luminometer LB960 (Berthold).

### 8. MMC Sensitivity Test

MMC-sensitivity test was performed on FA fibroblasts using a flow-cytometry based method, according to (35). Briefly, FA fibroblasts were plated in 24 multi-well plate at 10⁵ cells per well. MMC (Sigma Aldrich) was added at different concentrations and after 72 hours, cells were washed, trypsinized and harvested. Propidium iodide (PI, Sigma Aldrich) was added at a final concentration of 10 mg/mL in PBS-FBS, and the fluorescence was immediately analyzed by flow cytometry after gating of the cells by standard two-parameter forward scatter (FSC; size) and side scatter (SSC; granularity), using a FACSCalibur Flow Cytometer and CellQuest analysis system (BD Biosciences). The fraction of dying cells, which allows cellular permeabilization and PI uptake was measured by a shift on FL2. By including healthy and FA control cases in the experiment, comparison of the cell sensitivity to an increasing concentration of MMC clearly discriminates the FA phenotype

### 9. Detection of Click-Melphalan and Click-Mono Melphalan in Cells by Microscopy

HeLa cells were initially seeded onto coverslips in 6-well cluster plates at 300,000 cells per well and allowed to attach 4 hours. Cells were then treated with 1 μM of Clickable molecules or DMSO vehicle for 120 min and washed with PBS. Cell samples were then pre-extracted with ice-cold CSK buffer (100 mM NaCl, 10 mM HEPES pH 7.8, 3 mM MgCl₂, 300 mM sucrose, 0.5% Triton X-100) for 2 min on ice to remove non-chromatin bound protein and then fixed with 4% paraformaldehyde in PBS for 10 min at room temperature. Each sample was then washed with 3% FBS in PBS and then incubated with a click reaction mixture (50 μM Alexa fluor azide 647, 1 mM copper (II) sulfate, 100 mM sodium ascorbate, 100 mM Tris-HCl pH 8.0) at room temperature for 30 min. Cells were then washed using 3×10 min 3% FBS in PBS rinses with mild agitation and then mounted onto glass slides in VectaShield containing 1 μg/mL DAPI. Stained samples were captured and analyzed on a zeiss microscope equipped with a 40×/0.5 NA Plan Apo objective.

### 10. Detection of Click-Melphalan and Click-Mono Melphalan in Cells by Flow Cytometry

HeLa, Jurkat and FA or XP primary cells (10⁶ per dish) were seeded into 10 cm culture dishes and incubated overnight in culture medium. The following day, samples were treated with 0.5 μM click-Melphalan (Example 1), 0.5 μM click-MonoMelphalan (Example 2) or DMSO vehicle for 1 h. After treatment, cells were PBS-washed and released into medium. At designated time points, samples were harvested and fixed in EtOH 70%. Fixed cells where incubated in a click reaction buffer (50 μM Alexa fluor 647 azide, 1 mM copper (II) sulfate, 100 mM sodium ascorbate, 100 mM Tris-HCl pH 8.0) for 30 min at room temperature and then washed extensively using 3×5% FBS in PBS rinses to remove unbound reporter. Samples were stained with 5 μg/mL propidium iodide in 3% FBS in PBS and then analyzed using an LSR Fortessa flow cytometer (BD Biosciences, San Jose, CA). Ten thousand events were collected for each sample and gated using forward-versus side-scatter to eliminate cell debris and doublets. The data were analyzed using FlowJo software version 7.6.5 (Tree Star, San Carlos, CA).

### 11. Dual Parameter Plot Assay

HeLa and HeLa FANCD2 KO cells were treated with click-melphalan or click-mono-melphalan. Detection was done with Alexa Fluor 647 Click-iT Plus (Thermo Fisher) according to manufacture recommendations and DNA was co-stained with propidium iodide. Data were collected and analyzed using LSR Fortessa flow cytometer (BD Biosciences, San Jose, CA) using 635 nm excitation and a 660/20 nm bandpass emission filter for detection of the Alexa Fluor 647 picolyl azide and 488 nm excitation and a 610/20 bandpass emission filter for detection of the propidium iodide.

### 12. FA Patients Primary Cells

FA patient skin fibroblast cells were cryopreserved at Saint-Louis Hospital (Paris, France). Informed consents were obtained from the patients and/or their relatives and IRB approval from the INSERM was given under the number 12-078. Positive FA diagnosis in these patients had been performed using standard criteria including hypersensitivity to interstrand crosslinker agents in mononuclear blood cells and skin fibroblasts (36) (35). Biallelic germline FANCA (patient EGF400) or FANCG (patient EGF390) gene mutations were further identified by sequencing fibroblast DNAs. Control, non-FA cells were obtained with informed consent from unrelated non-FA individuals.

### 13. XP Patient Primary Cells

The primary fibroblast cells from two known XP patients (LXP82 and LXP924), who have a homozygous deleterious XPC mutation (XPC c.1643_1644 delTG; p.Val548AlafsX572) (37), were cryopreserved at Saint-Louis Hospital in Paris, France. Informed consent was obtained from either the patients or the parents of minor children. These fibroblast cells did not exhibit MMC hypersensitivity or abnormal FANCD2 monoubiquitination (not shown).

### 14. Diagnosis of FA Patients

The diagnosis of FA was based on clinical data, including personal and familial history, and physical examination, together with chromosome breakage test and FANCD2 detection.

Chromosome breakage tests were done as previously described (Pinto et al., Haematologica, 2009, 94, 487-495). Fresh PBLs were stimulated by PHA for 24 hours and further incubated with and without DNA damaging agent for 48 hours. We used the nitrogen mustard mechlorethamine (Caryolysine; Synthelabo, Le Plessis Robinson, France) freshly diluted at a final concentration of 0.05 μg/mL. Examination of 100 mitoses (50 with and 50 without DNA damaging agents) allowed the scoring of chromatid breaks and chromosome breaks as one break, and chromosome rearrangements and radials as 2 breaks; chromatid gaps were not scored. Results were compared with healthy controls and positive controls (FA patients) run in parallel. Cytogenetic diagnosis of FA is based on the combined analysis of several criteria: (1) the mean number of breaks per metaphase in nitrogen mustard (NM)-exposed cells (a mean number greater than 2 suggests FA); (2) the ratio of this mean between NM-exposed cells and nonexposed cells (a ratio greater than 10:1 is required); (3) the number of NM-dependent breaks per aberrant mitosis (5 or more breaks in several cells strongly suggests FA); and (4) the percentage of mitoses with break(s) in NM-exposed cells (a minimum of 20% of aberrant mitoses is required). If all criteria are fulfilled, FA is diagnosed. For FANCD2 detection, 30 μg of proteins from PHA-stimulated peripheral blood lymphocytes (PBLs), or 50 μg from growing nonconfluent fibroblasts, were loaded in a 80 mm×80 mm preloaded 3.8% Nu-PAGE Novex Tris-Acetate gel (Invitrogen). Proteins were migrated for 85 minutes using a Xcell SureLock Mini-Cell tank (Invitrogen) then transferred onto nitrocellulose membrane using the Xcell II Blot module (Invitrogen). After blocking for 2 hours or overnight in 0.5% milk, anti-FANCD2 (Abcam) was used at 1:1000 for 1 hour. Detection was performed using horseradish peroxide (HRP)-linked secondary antibodies with the ECL PLUS kit (Amersham Biosciences).

### Results

Modification for Click Chemistry does not Change the Level of Melphalan-Induced ICLs in Cells

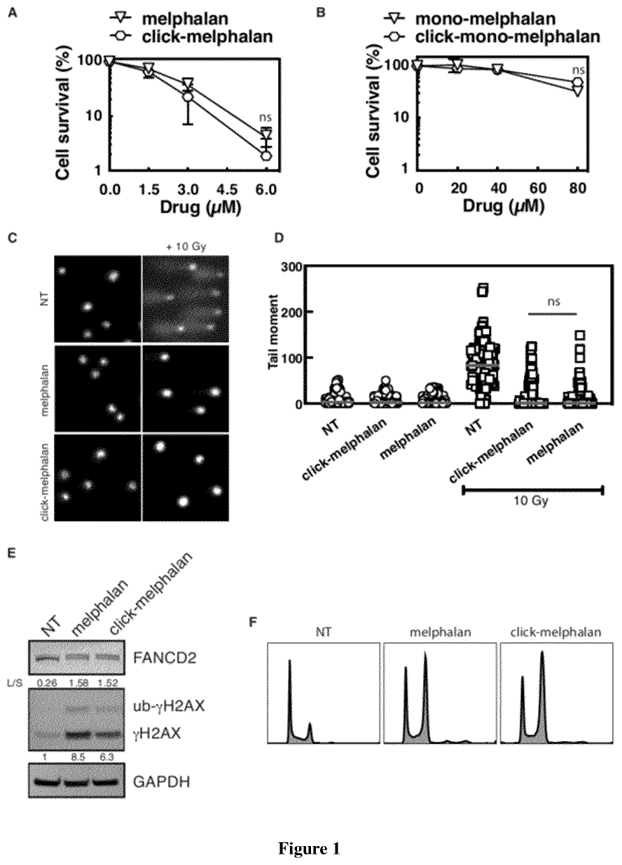

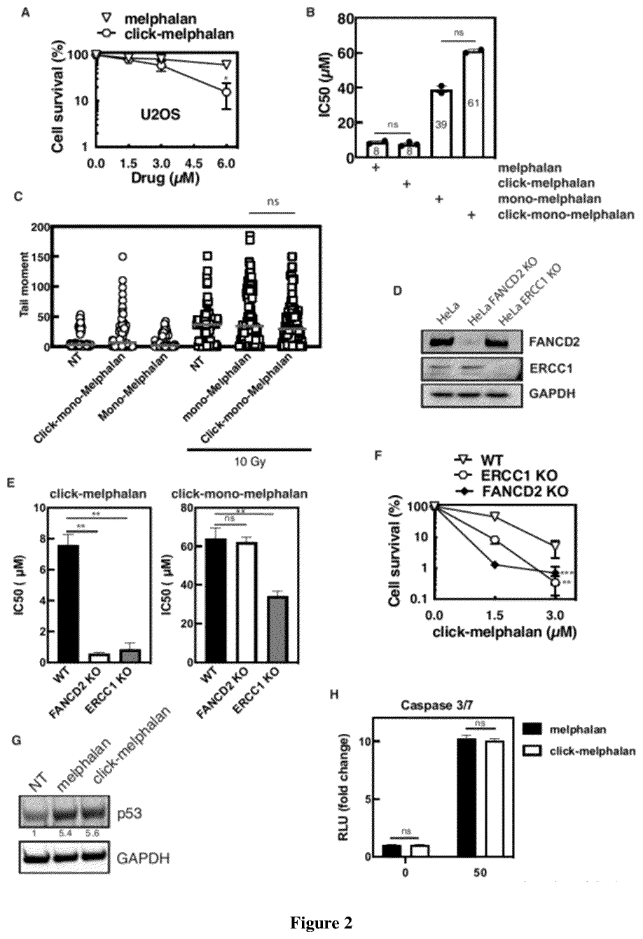

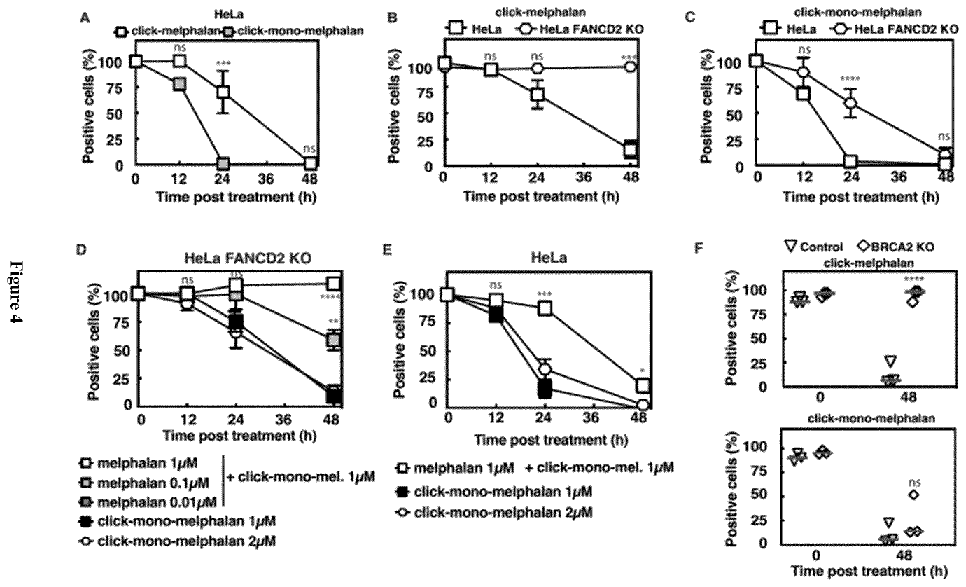

To determine if the addition of an alkyne groups on melphalan or Mono melphalan (Comparative Example 1) could affect the activity of the parent molecule, clonogenic assay was first used to compare the toxicity of compounds of the invention with the respective native compounds on HeLa cells (FIG. 1) and U2OS cells (FIG. 2A). Treatment of cells with both Melphalan and click-Melphalan (Example 1) (FIG. 1A) or Mono-melphalan (Comparative Example 1) and click-Mono-melphalan (Example 2) (FIG. 1B) similarly reduces the number of colonies formed with no significative differences. This was further confirmed by the measurement of IC50 activity (FIG. 2B). While the expected higher toxicity of melphalan compared to mono-melphalan reflects its unique ability to generate ICLs, the absence of differences in toxicity between Melphalan and click-Melphalan (Example 1) suggests that they produce a similar amount of DNA ICLs. A modified alkaline comet assay has previously been applied for the detection of DNA ICLs in cells. Similarly, this assay was used to confirm the equivalent capacity of click-melphalan (Example 1) to induce ICLs. In the absence of IR, neither Melphalan, click-Melphalan (Example 1), mono-melphalan (Comparative Example 1) nor click-mono-melphalan (Example 2) generated a visual comet score beyond the vehicle-treated control (FIG. 1C, 1D, 2C). IR treatment induced a strong increase in the “tail moment” of the comet, a measure of induced DNA damage. A pre-treatment with either melphalan or click-melphalan resulted in a large decrease in the IR-induced tail moment, while addition of Mono-melphalan (Comparative Example 1) and click-Mono-melphalan (Example 1) did not (FIG. 1C, 1D). As the reversal of the tail moment by Melphalan or click-Melphalan (Example 1) reflects the formation of ICLs and no significant difference was observed between Melphalan or click-Melphalan (Example 1), it can be concluded that the modification for Click chemistry does not change the level of melphalan-induced ICLs in cells. Accordingly, cells deficient for ERCC1 or FANCD2 (FIG. 4D), two proteins involved in ICL repair, are sensitive to click-melphalan (FIG. 2E, 2F). It was then tested whether other known responses of cells to melphalan treatment where also unaltered by the Click-Melphalan modification. Similar ubiquitination of FANCD2, phosphorylation of γH2AX, G2 arrest (FIG. 1F) or activation of p53 and caspase activity (FIG. 2G, 2H) where observed in response to both the parent and modified molecules, demonstrating that melphalan and click-melphalan induce an equivalent biological response.

DNA adducts induced by click-melphalan and click-mono-melphalan can be detected with an azide-tagged fluorescent reporter in cells via click chemistry

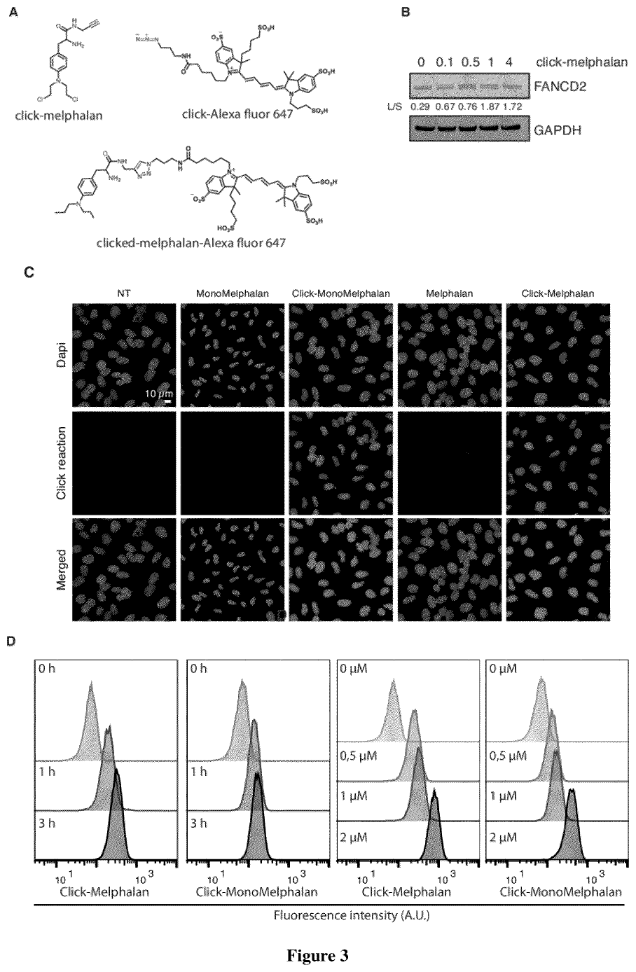

To establish whether click-Melphalan (Example 1) and click-Mono-melphalan (Example 2) can be used to monitor DNA lesions induced by both molecules, click-Melphalan (Example 1) and click-Mono-melphalan (Example 2) were conjugated with a fluorescent reporter in cells (FIG. 3A). Monitoring FANCD2 mono ubiquitination by western blot determined that 0.5 μM is the minimum concentration of click-melphalan (Example 1) required to detect a robust activation of the ICL repair pathway (FIG. 3B). HeLa cells were then exposed to 0.5 μM of click-Melphalan (Example 1), click-Mono-melphalan (Example 2) or unmodified melphalan. After treatment, whole cell samples were pre-extracted to remove non-chromatin bound molecules, fixed and then subjected to a click reaction mixture to enable ligation of the azide-tagged fluorescent reporter to click-Melphalan (Example 1) and click-Mono-melphalan (Example 2) adducts in cells. Nuclei were co-stained with DAPI prior analysis using confocal microscopy. Control samples exposed to non-modified molecules do not present significant levels of fluorescence indicating that the fluorescent reporter was not ligated to the DNA by the click reaction in the absence of clickable molecules (FIG. 3C). Upon treatment with click-Melphalan (Example 1) and click-Mono-melphalan (Example 2), fluorescence could be detected in cells (FIG. 3C), demonstrating that the post-labeling strategy is effective for in-cell detection of click-Melphalan (Example 1) and click-Mono-melphalan (Example 2) induced DNA lesions. The red fluorescent staining profile (middle panel, column 3-5, FIG. 3C) exhibited essentially the same staining pattern as the DNA content (Top panel, FIG. 3C), indicating that the red fluorescent reporter was conjugated to DNA containing covalent melphalan or mono-melphalan (Comparative Example 1) adducts. The next step was then to determine if detection by flow cytometry can be used to monitor Click-Melphalan (Example 1) or Click-Mono-Melphalan (Example 2) induced DNA lesions. After treatment with click-Melphalan (Example 1) and click-Mono-Melphalan (Example 2), HeLa cells were fixed, and samples were subjected to a click reaction mixture with azide-tagged fluorescent reporters. Extending the time of treatment increases the level of fluorescence, indicating an increased level of DNA lesions (FIG. 3D). Various concentrations of Click-Melphalan (Example 1) or Click-Mono-Melphalan (Example 2) were then applied, and it was determined that a fluorescent signal was detectable from 0.5 μM and increasing up to 2 μM (FIG. 3D). Collectively, these data validate Click-Melphalan (Example 1) and Click-Mono-Melphalan (Example 2) as functional clickable probes suitable to quantify DNA lesions in cells, with the ability to monitor their repair.

### The Presence of ICL Inhibits Monoadduct Repair

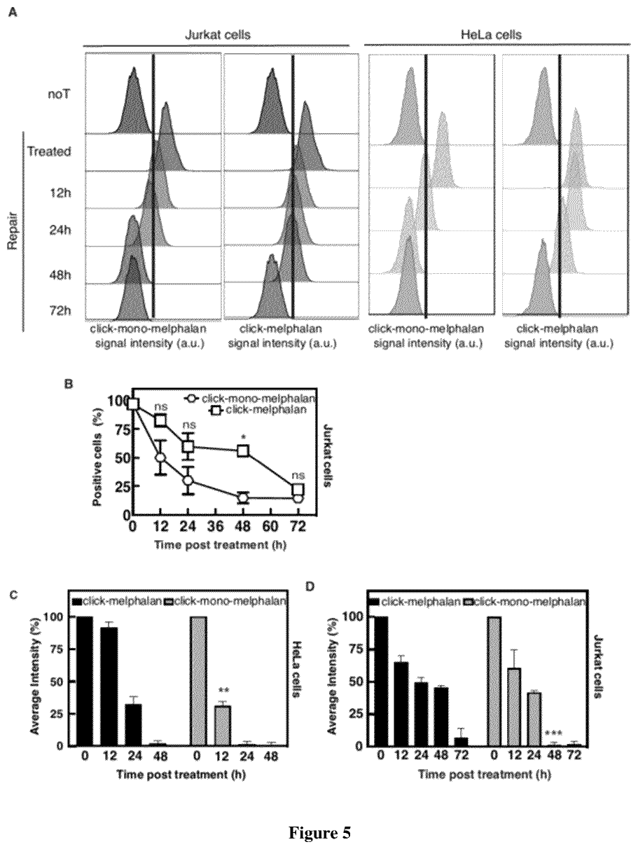

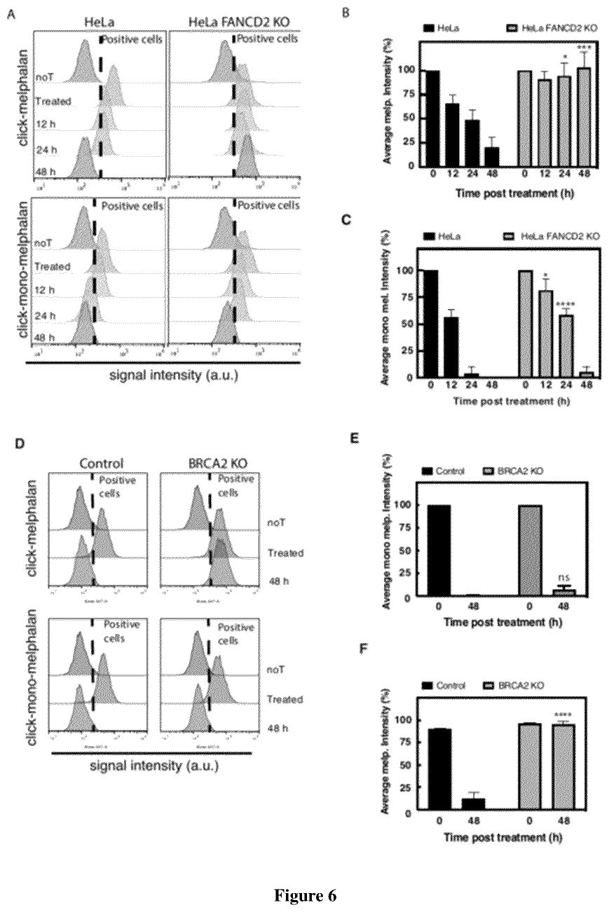

To quantitatively estimate the repair of mono-melphalan- or melphalan-induced lesions, azide-tagged fluorescent reporter were clicked to unsynchronized HeLa or Jurkat cells at different times after a pulse treatment with clickable molecules. The quantification of cells positive for lesions indicates that mono-melphalan-induced lesions are repaired faster that melphalan-induced lesions in both cell lines (FIG. 4A, 5A, 5B). Quantification of the median fluorescence intensity also confirmed a more efficient repair of lesions induced by click-mono-melphalan (FIG. 5C, 5D). It was then investigated whether click-melphalan could be used to detect an ICL repair defect using HeLa FANCD2 KO cells. While most of WT HeLa cells have repaired melphalan-induced lesions after 48 hours, all HeLa FANCD2 KO cells remained positive to melphalan (FIG. 3B, 6A top panel) with no reduction of the median fluorescence intensity (FIG. 6B). Surprisingly, a delay in the repair of monoadducts induced by mono-melphalan is observed at 24 hours in HeLa FANCD2 KO cells compared to HeLa cells but lesions are fully repaired in both cell lines at 48 hours (FIG. 3C, 6A lower panel, 6C). Considering that melphalan induces over 90% of monoadducts and that monoadducts are efficiently repaired in HeLa FANCD2 KO cells at 48 hours, it is thus surprising to not detect any repair of the lesions induced by melphalan in the absence of FANCD2. One possibility could be that the presence of unrepaired ICLs inhibits the repair of monoadducts. To test this hypothesis, FANCD2 KO cells were treated with both melphalan and click-mono-melphalan. In this configuration, since only click-mono-melphalan is clickable, only the monoadducts induced by click-mono-melphalan are detectable. While the addition of 0.01 μM of melphalan does not impact monoadduct repair, a decrease is observed with 0.1 μM and a complete inhibition is seen with 1 μM melphalan (FIG. 3D). The repair inhibition is not the mere consequence of a higher level of monoadducts induced by the addition of melphalan, since similar kinetics of repair were observed for lesions induced by 1 or 2 μM of click-mono-melphalan (FIG. 3D). Hence, it is concluded that the presence of unrepaired ICLs due to FANCD2 deficiency inhibits monoadduct repair (FIG. 3D).

Consistent with this hypothesis, the repair of monoadducts induced by click-mono-melphalan (Example 2) in WT HeLa cells is inhibited by the addition of non-clickable melphalan during the first 24 h of recovery (FIG. 3E), which corresponds to the time necessary to detect a significant ICL repair (FIG. 3A).

### ICLs are not Repaired in BRCA2-Deficient Cells

We next assessed the role of homologous recombination (HR) pathway, which is important for the repair of ICL downstream of FANCD2, using a BRCA2 KO RPE1 cell line. Similarly to FANCD2-deficient cells, click-mono-melphalan-induced lesions were repaired 48 hours post-treatment with no significant differences from the parental cells (FIG. 3F, 6D, 6E). However, BRCA2-deficient cells displayed a strong defect in the repair of click-melphalan-induced lesions (FIG. 3F, 6D, 6F). These data demonstrate that the method of the invention is an ideal set-up to examine the involvement of various DNA repair proteins in mono-adduct and ICL repair.

### ICLs are Repaired in Late S/G2 Phase by the FA Pathway

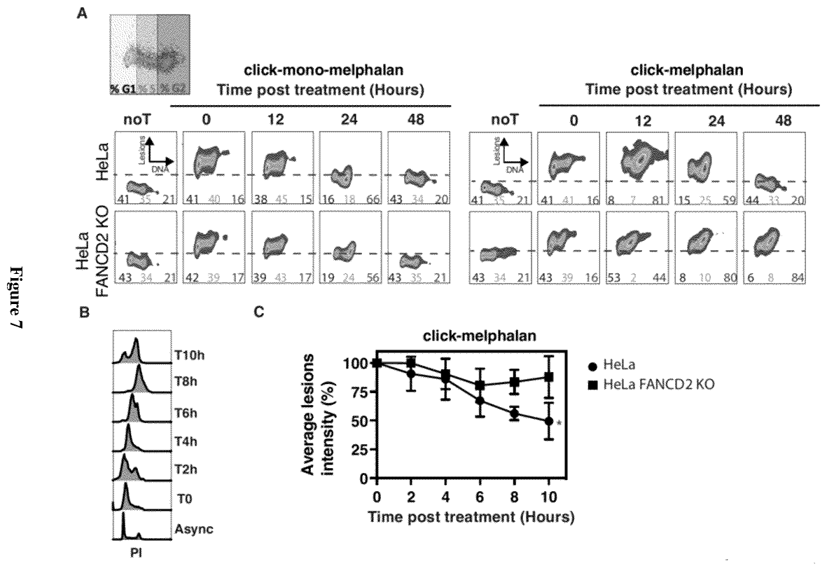

To gain insight into the timing of repair during the cell cycle, we combined click chemistry to label the lesions with propidium iodide to label DNA in HeLa and HeLa FANCD2 KO cell lines that are deficient for the FA pathway. As previously observed in FIG. 3, the repair of DNA lesions induced by mono-melphalan (Comparative Example 1) is slightly delayed in HeLa FANCD2 KO at 24 h compared to HeLa cells. The delay is observed in all cells independently of their DNA content (FIG. 7A left panel). Significant differences between HeLa and HeLa FANCD2 KO cell lines in the repair of melphalan-induced lesions were also detected. At 12 h post treatment, most of the HeLa positive cells are blocked in late S-G2 and a decrease in fluorescence is observed at 24 h when cells are still blocked in late S-G2 phase with a full repair at 48 h (FIG. 7A right panel, top lane) while FANCD2 KO cells remained blocked in late S-G2 phase until 48 h with unrepaired DNA lesions (FIG. 7A right panel bottom lane). These results suggest that melphalan-induced ICLs are normally repaired during late S phase in wild-type cells, in a FANCD2-dependent manner. To confirm this, cells were synchronized at the G1/S boundary using a double thymidine block (FIG. 7B, 8A). Before release into normal medium cells were exposed to click-melphalan (Example 1) and the level of lesions in cells was monitored every 2 h using click chemistry. Under these conditions, around 50% of lesions induced by click-melphalan (Example 1) are repaired in HeLa cells 10 h after release in S phase while no significant repair is observed in HeLa FANCD2 KO cells (FIG. 7C, 8B). Our data agree with current models of ICL repair in late S phase by the FA pathway (25) and demonstrate that click-melphalan (Example 1) can be used to monitor ICL repair during the cell cycle.

### Click-Melphalan can be Used to Identify ICL Repair Deficiency in FA Patient Cells

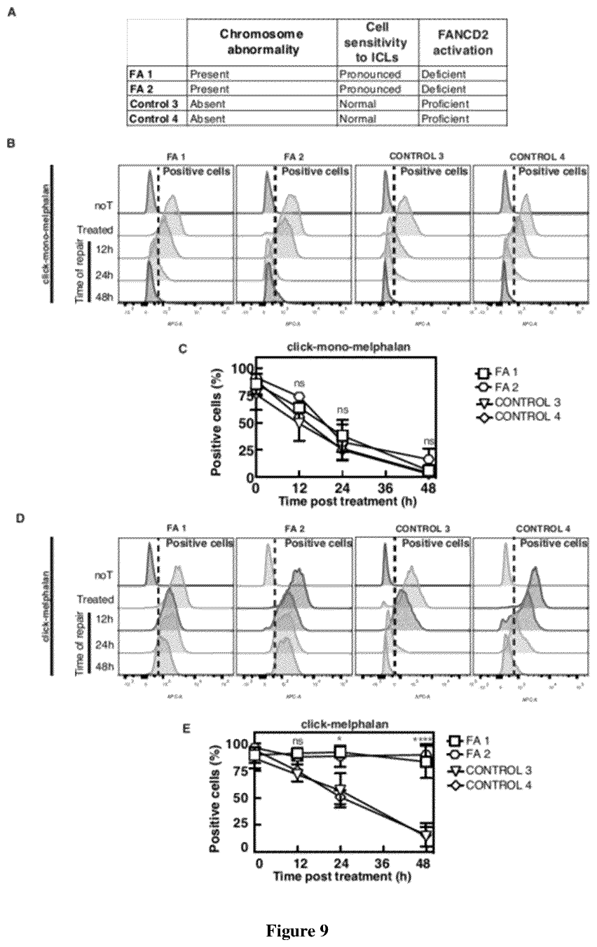

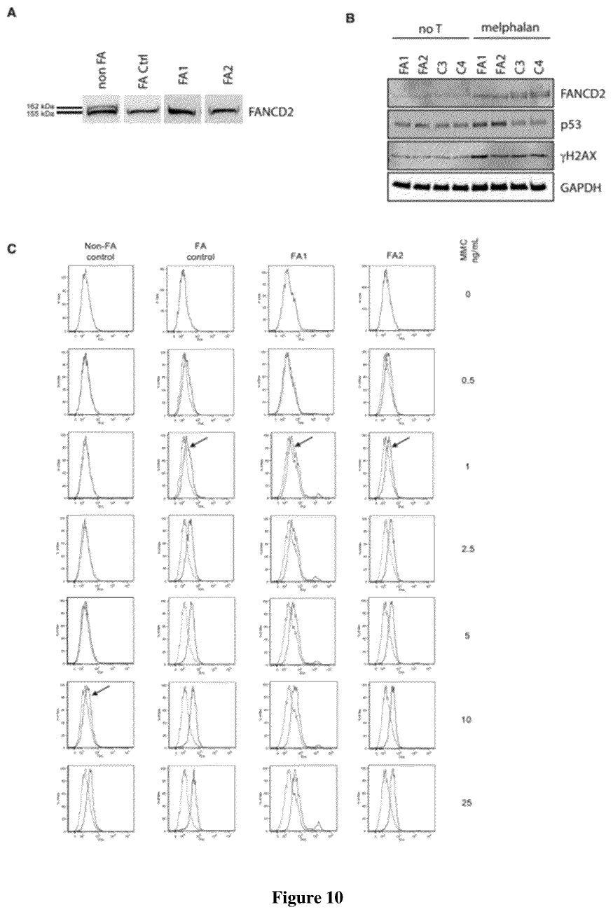

To determine whether monitoring ICL repair with click chemistry could be used to diagnose FA, primary fibroblast cells from two FA patients were studied, one FANCA (FA1) and the other FANCG (FA2) (patients EGF400 #1 and EGF390 #2, respectively), and compared to two non-FA subjects (C3 and C4). FA cells but not the control cells displayed a clear hypersensitivity to ICLs and no FANCD2 monoubiquitination (summarized in FIG. 9A, 10). The repair of lesions induced by mono-melphalan (Comparative Example 1) or melphalan using the compounds of the invention in the fibroblast cells from these 4 subjects was then monitored. No significant differences in the reduction of signal during repair of lesions induced by mono-melphalan (Comparative Example 1) were observed between the FA and non-FA cells (FIG. 9B) and all cells were negative for click-mono-melphalan after 48 hours (FIG. 9C). This result demonstrates that the FA cells do not display differences in their repair capacity towards DNA monoadducts. In striking contrast, lesions induced by melphalan persisted in the FA patient cells up to 48 h compared to non-FA (FIG. 9D) with less than 25% cells from subject C3 and C4 still positive for melphalan compared to nearly 100% in the FA patients FA1 and FA2 (FIG. 9E). To further evaluate the potential of the method of the invention to diagnose FA using lymphocytes, it was applied to cryopreserved peripherical blood samples corresponding to the same patients previously tested. Due to limited quantity of cells available, lesions were only quantified 48 hours post treatment. In concordance with the results obtained in fibroblasts, PHA-stimulated lymphocytes from both FA patients were able to repair mono-melphalan—but not melphalan—induced lesions (FIG. 10).

Since the defect in repair is observed with melphalan but not with mono-melphalan, it can be concluded that both fibroblasts and lymphocytes from FA patients are deficient in ICL repair. These data demonstrate that the compounds of the invention are able to support the current diagnostic regimen for FA patient by quickly identifying their defect in ICL repair.

### Click-Mono-Melphalan and Click-Melphalan can be Used to Identify Deficiencies in Xeroderma Pigmentosum Patient Cells

Xeroderma pigmentosum (XP) is a rare inherited disorder due to a defective Nucleotide excision repair (NER) pathway. NER removes bulky DNA lesions such as those formed by UV light or some cancer chemotherapeutic agents including cisplatin and melphalan.

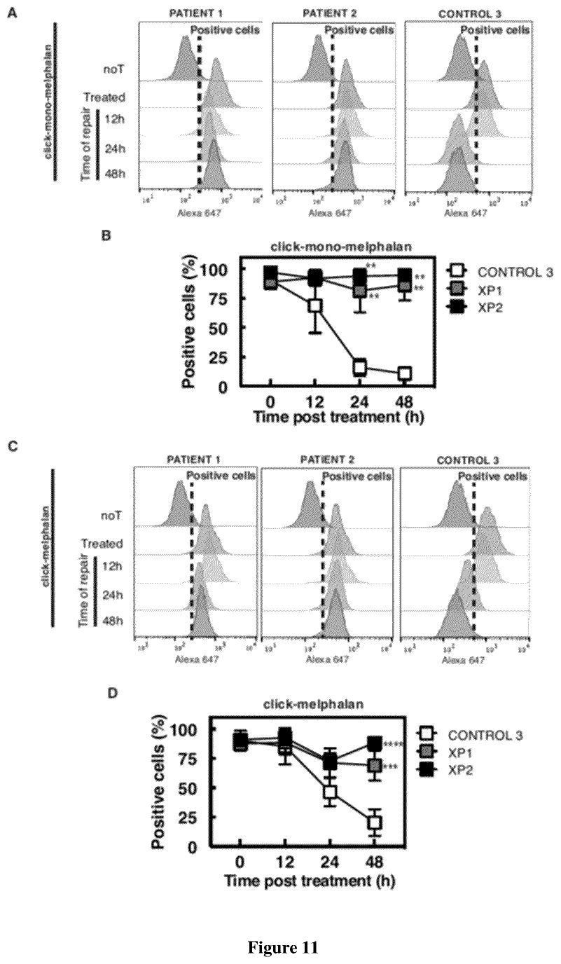

Building on our work detecting ICL repair defects in FA patient cells, the ability of the compounds of the invention to also detect NER defects in fibroblast cells from two patients with XP was evaluated. Lesions induced by mono-melphalan persisted in the XP patient cells up to 48 hours compared to non-XP cells (FIG. 11A, 11B), with nearly 0% of cells from subject C3 still positive for mono-melphalan compared to nearly 100% in the XP patients XP1 and XP2 (FIG. 11B). Similar defects in repair were detected for lesions induced by melphalan, with 25% of cells from subject C3 still positive for melphalan compared to nearly 75% in the XP patients XP1 and XP2 (FIG. 11C, 11D).

These data demonstrate that the compounds of the invention have the ability to evidence syndromes other than FA, in particular Xeroderma pigmentosum, due to defects in pathways involved in the repair of melphalan- or mono-melphalan-induced lesions.

1. A compound comprising a general of Formula I: 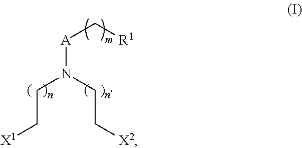
a pharmaceutically acceptable salt, or a pharmaceutically acceptable solvate thereof,
wherein
R¹ is C≡CH or N₃;
m is an integer ranging from 1 to 6;
A is selected from 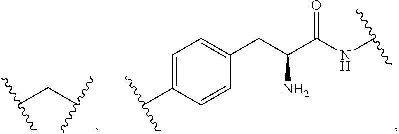 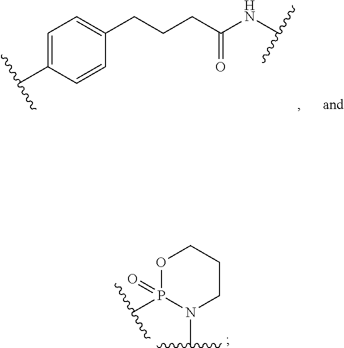 [SMILES: *NC(=O)CCCc1ccc(*)cc1 ; *N1CCCOP1(*)=O]
n and n′ are integers independently ranging from 1 to 6;
X¹ is a first halogen; and
X² is a second halogen or OH, wherein the first halogen and the second halogen are the same or different.

2. The compound according to claim 1, having the formula II: 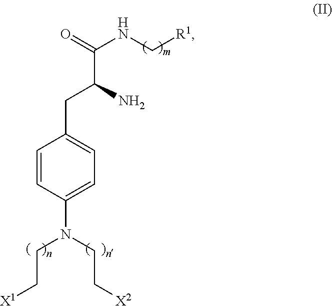
or a pharmaceutically acceptable salt, or a pharmaceutically acceptable solvate thereof.

3. The compound according to claim 1, wherein R¹ is C≡CH.

4. The compound according to claim 1, wherein m is an integer ranging from 1 to 3.

5. The compound according to claim 1, wherein n and n′ are 1.

6. The compound according to claim 1, wherein X¹ is Cl.

7. The compound according to claim 1, wherein X² is Cl or OH.

8. The compound according to claim 1, selected from the group consisting of:
(S)-2-amino-3-(4-(bis(2-chloroethyl)amino)phenyl)-N-(prop-2-yn-1-yl)propanamide;
(S)-2-amino-3-(4-((2-chloroethyl) (2-hydroxyethyl)amino)phenyl)-N-(prop-2-yn-1-yl)propanamide;
(S)-2-amino-3-(4-(bis(2-chloroethyl)amino)phenyl)-N-(pent-4-yn-1-yl)propanamide; and
(S)-2-amino-N-(3-azidopropyl)-3-(4-(bis(2-chloroethyl)amino)phenyl)propanamide.

9. A kit comprising:
at least one compound according to claim 1, and
at least one label bearing a group complementary to the R¹ group of said at least one compound for a “click chemistry” reaction, the label being in particular a fluorescent label or a biotinylated label.

10. (canceled)

11. An in-vitro or ex vivo method for detecting changes in cells, the method comprising:
(a) having at least one cell,
(b) contacting said cell with at least one compound of formula (I) according to claim 1 under conditions allowing said compound of formula (I) to induce DNA crosslinks in the cell,
(c) contacting the cell obtained in step (b) with at least one label bearing a group which is complementary for a click-chemistry reaction to the R¹ group of the said compound of formula (I), under conditions allowing the reaction of click-chemistry between the said R¹ group and the complementary group,
(d) detecting the label in the cell obtained at step (c).

12. The method of claim 11, further comprising
(d) measuring the labeling in the cell obtained at step (c), and
(e) optionally comparing the labeling measured at step (d) to a reference level.

13. The method according to claim 11, wherein the label is a fluorescent label.

14. The method according to claim 11, wherein the patient cell is from a Fanconi Anemia (FA) patient, an xeroderma pigmentosum (XP) patient, an acute myeloid leukemia (AML) patient, a head-and-neck cancer patient, an ovarian cancer patient, or a breast cancer patient.

15. The compound according to claim 2, wherein R¹ is C≡CH.

16. The compound according to claim 15, wherein m is an integer ranging from 1 to 3.

17. The compound according to claim 16, wherein n and n′ are 1.

18. The compound according to claim 17, wherein X¹ is Cl and X² is Cl or OH.

19. The kit of claim 9, wherein A is 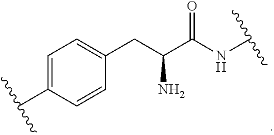 [SMILES: *NC(=O)[C@@H](N)Cc1ccc(*)cc1]
